SEQUENCE LISTING (3 sequences)

SEQ ID NO: 1 - DNA, 3958 bp, Homo sapiens
ctactccaggctgtgttcagggcttggggctggtggagggaggggcctgaaattccagtgtgaaaggctgagatgggcccgaggcccctggcctatgtccaagccatttcccctctcaccagcctctccctggggagccagtcagctaggaaggaatgagggctccccaggcccacccccagttcctgagctcatctgggctgcagggctggcgggacagcagcgtggactcagtctcctagggatttcccaactctcccgcccgcttgctgcatctggacaccctgcctcaggccctcatctccactggtcagcaggtgacctttgcccagcgccctgggtcctcagtgcctgctgccctggagatgatataaaacaggtcagaaccctcctgcctgtctgctcagttcatccctagaggcagctgctccaggtaatgccctctggggaggggaaagaggaggggaggaggatgaagaggggcaagaggagctccctgcccagcccagccagcaagcctggagaagcacttgctagagctaaggaagcctcggagctggacgggtgccccccacccctcatcataacctgaagaacatggaggcccgggaggggtgtcacttgcccaaagctacatagggggtggggctggaagtggctccaagtgcaggttcccccctcattcttcaggcttagggctggaggaagccttagacagcccagtcctaccccagacagggaaactgaggcctggagagggccagaaatcacccaaagacacacagcatgttggctggactggacggagatcagtccagaccgcaggtgccttgatgttcagtctggtgggttttctgctccatcccacccacctccctttgggcctcgatccctcgcccctcaccagtcccccttctgagagcccgtattagcagggagccggcccctactccttctggcagacccagctaaggttctaccttaggggccacgccacctccccagggaggggtccagaggcatggggacctggggtgcccctcacaggacacttccttgcaggaacagaggtgccatgcagccccgggtactccttgttgttgccctcctggcgctcctggcctctgcccgtaagcacttggtgggactgggctgggggcagggtggaggcaacttggggatcccagtcccaatgggtggtcaagcaggagcccagggctcgtccataggccgatccaccccactcagccctgctctttcctcaggagcttcagaggccgaggatgcctcccttctcagcttcatgcagggctacatgaagcacgccaccaagaccgccaaggatgcactgagcagcgtgcaggagtcccaggtggcccagcaggccaggtacacccgctggcctccctccccatcccccctgccagctgcctccattcccacccacccctgccctggtgagatcccaacaatggaatggaggtgctccagcctcccctgggcctgtgcctcttcagcctcctctttcctcacagggcctttgtcaggctgctgcgggagagatgacagagttgagactgcattcctcccaggtccctcctttctccccagagcagtcctagggcgcgccgttttagccctcatttccattttcctttcctttccctttctttccctttctatttctttctttctttctttctttctttctttctttctttctttctttctttctttctttctttctttcctttctttctttcttttcttctttctttctttcctttctttctctttctttctttctttctttcctttttctttctttccctctcttcctttctctctttctttcttcttcttttttttttaatggagtctccctctgtcacccaggctggagtgcagtggtgccatctcggctcactgcaacctccgtctcccgggttcaacccattctcctgcctcagcctcccaagtagctgggattacaggcacgcgccaccacacccagctaatttttgtatttttagcagagatggggtttcaccatgttggccaggttggtcttgaattcctgacctcaggggatcctcctgcctcggcctcccaaagcgctgggattacaggcatgagccactgcgcctggccccattttccttttctgaaggtctggctagagcagtggtcctcagcctttttggcaccagggaccagttttgtggtggacaatttttccatgggccagcggggatggttttgggatgaagctgttccacctcagatcatcaggcattagattctcataaggagccctccacctagatccctggcatgtgcagttcacaacagggttcacactcctatgagaatgtaaggccacttgatctgacaggaggcggagctcaggcggtattgctcactcacccaccactcacttcgtgctgtgcagcccggctcctaacagtccatggaccagtacctatctatgacttgggggttggggacccctgggctaggggtttgccttgggaggccccacctgacctaattcaagcccgtgagtgcttctgctttgttctaagacctggggccagtgtgagcagaagtgtgtccttcctctcccatcctgcccctgcccatcagtactctcctctcccctactcccttctccacctcaccctgactggcattagctggcatagcagaggtgttcataaacattcttagtccccagaaccggctttggggtaggtgttattttctcactttgcagatgagaaaattgaggctcagagcgattaggtgacctgccccagatcacacaactaatcaatcctccaatgactttccaaatgagaggctgcctccctctgtcctaccctgctcagagccaccaggttgtgcaactccaggcggtgctgtttgcacagaaaacaatgacagccttgacctttcacatctccccaccctgtcactttgtgcctcaggcccaggggcataaacatctgaggtgacctggagatggcagggtttgacttgtgctggggttcctgcaaggatatctcttctcccagggtggcagctgtgggggattcctgcctgaggtctcagggctgtcgtccagtgaagttgagagggtggtgtggtcctgactggtgtcgtccagtggggacatgggtgtgggtcccatggttgcctacagaggagttctcatgccctgctctgttgcttcccctgactgatttaggggctgggtgaccgatggcttcagttccctgaaagactactggagcaccgttaaggacaagttctctgagttctgggatttggaccctgaggtcagaccaacttcagccgtggctgcctgagacctcaataccccaagtccacctgcctatccatcctgccagctccttgggtcctgcaatctccagggctgcccctgtaggttgcttaaaagggacagtattctcagtgctctcctaccccacctcatgcctggcccccctccaggcatgctggcctcccaataaagctggacaagaagctgctatgagtgggccgtcgcaagtgtgccatctgtgtctgggcatgggaaagggccgaggctgttctgtgggtgggcactggacagactccaggtcaggcaggcatggaggccagcgctctatccaccttctggtagctgggcagtctctgggcctcagtttcttcatctctaaggtaggaatcaccctccgtaccctgccttccttgacagctttgtgcggaaggtcaaacaggacaataagtttgctgatactttgataaactgttaggtgctgcacaacatgacttgagtgtgtgccccatgccagccactatgcctggcacttaagttgtcatcagagttgagactgtgtgtgtttactcaaaactgtggagctgacctcccctatccaggccacctagccct

SEQ ID NO: 2 - DNA, 567 bp, Homo sapiens
aaccctcctgcctgtctgctcagttcatccctagaggcagctgctccaggaacagaggtgccatgcagccccgggtactccttgttgttgccctcctggcgctcctggcctctgcccgagcttcagaggccgaggatgcctcccttctcagcttcatgcagggttacatgaagcacgccaccaagaccgccaaggatgcactgagcagcgtgcaggagtcccaggtggcccagcaggccaggggctgggtgaccgatggcttcagttccctgaaagactactggagcaccgttaaggacaagttctctgagttctgggatttggaccctgaggtcagaccaacttcagccgtggctgcctgagacctcaataccccaagtccacctgcctatccatcctgcgagctccttgggtcctgcaatctccagggctgcccctgtaggttgcttaaaagggacagtattctcagtgctctcctaccccacctcatgcctggcccccctccaggcatgctggcctcccaataaagctggacaagaagctgctatgagtaaaaaaaaaaaaaaa

SEQ ID NO: 3 - DNA, 20 bp, Artificial sequence
  Synthetic oligonucleotide
agcttcttgtccagctttat

(19) United States
(12) Patent Application Publication
(10) Pub. No.: US 2024/0033280 A1
(43) Pub. Date: Feb. 1, 2024

(54) METHODS FOR REDUCING APOCIII EXPRESSION

(71) Applicant: IONIS PHARMACEUTICALS, INC., Carlsbad, CA (US)

(72) Inventors: Sotirios TSIMIKAS, Carlsbad, CA (US); Veronica J. ALEXANDER, Carlsbad, CA (US); Stanley T. CROOKE, Carlsbad, CA (US); Richard S. GEARY, Carlsbad, CA (US); Brett P. MONIA, Carlsbad, CA (US)

(21) Appl. No.: 18/247,567

(22) PCT Filed: Sep. 24, 2021
(86) PCT No.: PCT/US2021/052001

§ 371 Date: Mar. 31, 2023

Related U.S. Application Data
(60) Provisional application No. 63/087,095, filed on Oct. 2, 2020.

(51) Int. Cl. A61K 31/7125; A61P 3/06

(52) U.S. Cl. CPC A61K 31/7125; A61P 3/06

(57) ABSTRACT

Provided herein are methods of administering ISIS 678354 for ameliorating Familial Chylomicronemia Syndrome (FCS), Familial Partial Lipodystrophy (FPL), Severe Hypertriglyceridemia (SHTG), reducing APOCIII RNA, or reducing APOCIII protein in a human subject in need thereof. In certain instances, methods are useful for ameliorating at least one symptom of FCS, FPL, or SHTG. Such symptoms of FCS include, but are not limited to, severe elevations in chylomicrons and extremely elevated TG levels (always reaching well above 1000 mg/dL and not infrequently rising as high as 10,000 mg/dL or more) with episodes of abdominal pain, physical fatigue, difficulty thinking, diarrhea, recurrent acute pancreatitis, eruptive cutaneous xanthomata, and hepatosplenomegaly.

### SEQUENCE LISTING

The present application is being filed along with a Sequence Listing in electronic format. The Sequence Listing is provided as a file entitled BIOL0406WOSEQ_ST25.txt, created on Sep. 23, 2021, which is 8 KB in size. The information in the electronic format of the sequence listing is incorporated herein by reference in its entirety.

### FIELD

Provided herein are methods of administering ISIS 678354 for ameliorating Familial Chylomicronemia Syndrome (FCS), Familial Partial Lipodystrophy (FPL), Severe Hypertriglyceridemia (SHTG), reducing APOCIII RNA, or reducing APOCIII protein in a human subject in need thereof. In certain instances, methods are useful for ameliorating at least one symptom of FCS, FPL, or SHTG. Such symptoms of FCS include, but are not limited to, severe elevations in chylomicrons (severe chylomicronemia) and extremely elevated TG levels (always reaching well above 1000 mg/dL and not infrequently rising as high as 10,000 mg/dL or more; severe hypertriglyceridemia) with episodes of abdominal pain, physical fatigue, difficulty thinking, diarrhea, recurrent acute pancreatitis, eruptive xanthomas, lipemia retinalis, and hepatosplenomegaly.

### BACKGROUND

Familial Chylomicronemia Syndrome (FCS) is an inherited disease characterized by severe hypertriglyceridemia and chylomicronemia. It is a rare autosomal recessive disease that can be diagnosed either in childhood or adulthood. FCS is characterized by frequent and severe abdominal pain, repetitive colicky pain, repeated episodes of potentially fatal acute pancreatitis, and in children, can result in a failure to thrive (Familial Lipoprotein Lipase Deficiency. In GeneReviews. edited by Adam M P Pagon R A, Bird T D, et al. 1999-2011. Seattle, WA: University of Washington, Seattle; Etiology and risk of latescent plasma and severe hypertriglyceridemia. J Clin Lipidol 2011; 5: 37-44). Physical examination frequently reveals eruptive xanthomas, lipemia retinalis and hepatosplenomegaly, and plasma from patients appears latescent, interfering with determination of other laboratory parameters. Fasting plasma TG levels in FCS patients are typically 10-fold to 100-fold above normal (1,500 to 15,000 mg/dL), despite extreme dietary fat restriction (20 g or approximately 15-20% of daily calorie intake). Patients with FCS often present in infancy or childhood with recurrent episodes of abdominal pain or pancreatitis, eruptive xanthomas or hepatomegaly. The diagnosis of FCS is then established by genotyping or confirmation of very low or absent lipoprotein lipase (LPL) enzyme activity in post-heparin plasma. Patient having FCS or other familial disorders such as Familial Partial Lipodystrophy (FPL) may present with severe hypertriglyceridemia.

Apolipoprotein C-III (also called APOC3, APOC-III, ApoCIII, and APO C-III) is a constituent of HDL and of triglyceride (TG)-rich lipoproteins. Elevated ApoCIII levels are associated with elevated TG levels and diseases such as cardiovascular disease, metabolic syndrome, obesity and diabetes (Chan et al., Int J Clin Pract, 2008, 62:799-809; Onat et at., Atherosclerosis, 2003, 168:81-89; Mendivil et al., Circulation, 2011, 124:2065-2072; Mauger et al., J. Lipid Res, 2006. 47: 1212-1218; Chan et al., Clin. Chem, 2002. 278-283; Ooi et al., Clin. Sci, 2008. 114: 611-624; Davidsson et al., J. Lipid Res. 2005. 46: 1999-2006; Sacks et al., Circulation, 2000. 102: 1886-1892; Lee et al., Arterioscler Thromb Vase Biol, 2003. 23: 853-858). ApoCIII slows clearance of TG-rich lipoproteins by inhibiting lipolysis through inhibition of lipoprotein lipase (LPL) and through interfering with lipoprotein binding to cell-surface glycosaminoglycan matrix (Shachter, Curr. Opin. Lipidol, 2001, 12, 297-304).

Antisense technology is emerging as an effective means for reducing the expression of certain gene products and may prove to be uniquely useful in a number of therapeutic, diagnostic, and research applications for the modulation of ApoCIII. Antisense compounds targeting ApoCIII and associated methods for inhibiting ApoCIII have been previously disclosed (see e.g., U.S. Pat. Nos. 7,598,227, 7,750,141, PCT publication WO 2004/093783, PCT publication WO 2012/149495, PCT/US14/016546, and WO 2014/179626, all incorporated-by-reference herein).

### SUMMARY OF THE INVENTION

Provided herein are methods for ameliorating Familial Chylomicronemia Syndrome (FCS), Familial Partial Lipodystrophy (FPL), or Severe Hypertriglyceridemia (SHTG), and methods of reducing APOCIII RNA and/or APOCIII protein in a human subject in need thereof. In certain embodiments, methods comprise administering a therapeutically effective amount of a modified oligonucleotide. In certain embodiments, the modified oligonucleotide is ISIS 678354. In certain embodiments, the therapeutically effective amount is within the range of about 40 mg to about 100 mg. In certain embodiments, the therapeutically effective amount is about 50 mg. In certain embodiments, the therapeutically effective amount is about 80 mg. In certain embodiments, the therapeutically effective amount is administered once about every 4 weeks.

### DETAILED DESCRIPTION OF THE INVENTION

It is to be understood that both the foregoing general description and the following detailed description are exemplary and explanatory only and are not restrictive. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated-by-reference for the portions of the document discussed herein, as well as in their entirety.

### Definitions

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Where permitted, all patents, applications, published applications and other publications and other data referred to throughout in the disclosure are incorporated by reference herein in their entirety.

Unless otherwise indicated, the following terms have the following meanings:

As used herein, “2′-deoxyribonucleoside” means a nucleoside comprising a 2′-H(H) deoxyribosyl sugar moiety. In certain embodiments, a 2′-deoxyribonucleoside is a 2′-β-D deoxyribonucleoside and comprises a 2′-β-D-deoxyribosyl sugar moiety, which has the β-D configuration as found in naturally occurring deoxyribonucleic acids (DNA). In certain embodiments, a 2′-deoxyribonucleoside may comprise a modified nucleobase or may comprise an RNA nucleobase (uracil).

As used herein, “2′-MOE” means a 2′-OCH₂CH₂OCH₃ group in place of the 2′-OH group of a ribosyl sugar moiety. A “2′-MOE sugar moiety” is a sugar moiety with a 2′-OCH₂CH₂OCH₃ group in place of the 2′-OH group of a ribosyl sugar moiety. Unless otherwise indicated, a 2′-MOE sugar moiety is in the β-D configuration. “MOE” means O-methoxyethyl.

As used herein, “2′-MOE nucleoside” means a nucleoside comprising a 2′-MOE sugar moiety.

As used herein, “5-methyl cytosine” means a cytosine modified with a methyl group attached to the 5 position. A 5-methyl cytosine is a modified nucleobase.

As used herein, “about” means plus or minus 7% of the provided value.

As used herein, “administering” means providing a pharmaceutical agent to a human subject.

As used herein, “ameliorate” in reference to a treatment means improvement in at least one symptom relative to the same symptom in the absence of the treatment. In certain embodiments, amelioration is the reduction in the severity or frequency of a symptom, or the delayed onset or slowing of progression in the severity or frequency of a symptom.

As used herein, “adjudicated pancreatitis event” is a formally confirmed event of pancreatitis. Events of confirmed pancreatitis may be further classified as acute, chronic, or unknown type (i.e. indeterminate: events that may have met the definition of pancreatitis but were unable to be classified as either acute or chronic).

As used herein, “Adverse event” or “AE” can be any unfavorable and unintended sign (including an abnormal laboratory finding, for example), symptom, or disease temporally associated with the use of the medicinal (investigational) product, whether or not the AE is considered related to the medicinal (investigational) product. In certain embodiments, the medicinal (investigational) product is ISIS 678354.

As used herein, “APOCIII RNA” is the RNA expression product of the human gene, APOCIII.

As used herein, “APOCIII protein” is the protein expression product of APOCIII RNA.

As used herein, “ApoCIII”, “Apolipoprotein C-III” or “ApoC3” means any nucleic acid or protein sequence encoding ApoCIII. For example, in certain embodiments, an ApoCIII includes a DNA sequence encoding ApoCIII, a RNA sequence transcribed from DNA encoding ApoCIII (including genomic DNA comprising introns and exons), a mRNA sequence encoding ApoCIII, or a peptide sequence encoding ApoCIII.

As used herein, “ApoCIII nucleic acid” means any nucleic acid encoding ApoCIII. For example, in certain embodiments, an ApoCIII nucleic acid includes a DNA sequence encoding ApoCIII, a RNA sequence transcribed from DNA encoding ApoCIII (including genomic DNA comprising introns and exons), and a mRNA sequence encoding ApoCIII.

As used herein, “baseline” is defined as the average of all pre-dose measurements and/or assessments. In certain embodiments, “baseline is defined as the last assessment prior to the first dose of the drug. In certain embodiments, the drug is ISIS 678354.

As used herein, “diabetes mellitus” or “diabetes” is a syndrome characterized by disordered metabolism and abnormally high blood sugar (hyperglycemia) resulting from insufficient levels of insulin or reduced insulin sensitivity. The characteristic symptoms are excessive urine production (polyuria) due to high blood glucose levels, excessive thirst and increased fluid intake (polydipsia) attempting to compensate for increased urination, blurred vision due to high blood glucose effects on the eye's optics, unexplained weight loss, and lethargy.

As used herein, “diabetic dyslipidemia” or “type 2 diabetes with dyslipidemia” means a condition characterized by Type 2 diabetes, reduced HDL-C, elevated triglycerides (TG), and elevated small, dense LDL particles.

As used herein, “dose” means a quantity of a pharmaceutical agent administered.

As used herein, “dyslipidemia” refers to a disorder of lipid and/or lipoprotein metabolism, including lipid and/or lipoprotein overproduction or deficiency. Dyslipidemias can be manifested by elevation of lipids such as chylomicron, cholesterol and triglycerides as well as lipoproteins such as low-density lipoprotein (LDL) cholesterol.

As used herein, “hypercholesterolemia” means a condition characterized by elevated cholesterol or circulating (plasma) cholesterol, LDL-cholesterol and VLDL-cholesterol, as per the guidelines of the Expert Panel Report of the National Cholesterol Educational Program (NCEP) of Detection, Evaluation of Treatment of high cholesterol in adults (see, Arch. Int. Med. (1988) 148, 36-39).

As used herein, “hyperlipidemia” or “hyperlipemia” is a condition characterized by elevated serum lipids or circulating (plasma) lipids. This condition manifests an abnormally high concentration of fats. The lipid fractions in the circulating blood are cholesterol, low density lipoproteins, very low density lipoproteins, chylomicrons and triglycerides.

As used herein, “hypertriglyceridemia” means a condition characterized by elevated triglyceride levels. Hypertriglyceridemia is the consequence of increased production and/or reduced or delayed catabolism of triglyceride (TG)-rich lipoproteins: VLDL and, to a lesser extent, chylomicrons (CM).

As used herein, the term “internucleoside linkage” means the covalent linkage between contiguous nucleosides in an oligonucleotide. As used herein “modified internucleoside linkage” means any internucleoside linkage other than a phosphodiester internucleoside linkage. “Phosphorothioate internucleoside linkage” is a modified internucleoside linkage in which one of the non-bridging oxygen atoms of a phosphodiester internucleoside linkage is replaced with a sulfur atom.

As used herein, “loading dose” means a therapeutically effective amount of a pharmaceutical agent administered during an initial dosing phase during which steady state concentration of the pharmaceutical agent is achieved. “Initial loading dose” means the first loading dose administered. “Last loading dose” means the loading dose administered most recently prior to administering a first maintenance dose.

As used herein, “maintenance dose” means a therapeutically effective amount of a pharmaceutical agent administered during a dosing phase after steady state concentration of the pharmaceutical agent has been achieved.

As used herein, “nucleobase” means an unmodified nucleobase or modified nucleobase. An “unmodified nucleobase” is adenine (A), thymine (T), cytosine (C), uracil (U), or guanine (G). A “modified nucleobase” is group of atoms other than unmodified A, T, C, U, or G capable of pairing with at least one unmodified nucleobase. A “5-methyl cytosine” is a modified nucleobase. As used herein, “nucleobase sequence” means the order of contiguous nucleobases in a target nucleic acid or oligonucleotide independent of any sugar or internucleoside linkage modification.

As used herein, “nucleoside” means a compound comprising a nucleobase and a sugar moiety. The nucleobase and sugar moiety are each, independently, unmodified or modified. As used herein, “modified nucleoside” means a nucleoside comprising a modified nucleobase and/or a modified sugar moiety. “Linked nucleosides” are nucleosides that are connected in a contiguous sequence (i.e., no additional nucleosides are presented between those that are linked). As used herein, “oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides. As used herein, “modified oligonucleotide” means an oligonucleotide, wherein at least one nucleoside or internucleoside linkage is modified.

As used herein, “pharmaceutically acceptable carrier or diluent” means any substance suitable for use in administering to a human subject. Certain such carriers enable pharmaceutical compositions to be formulated as, for example, tablets, pills, dragees, capsules, liquids, gels, syrups, slurries, suspension, and lozenges for the oral ingestion by a human subject. In certain embodiments, a pharmaceutically acceptable carrier or diluent is sterile water, sterile saline, sterile buffer solution, or sterile artificial cerebrospinal fluid.

As used herein, “pharmaceutically acceptable salts” means physiologically and pharmaceutically acceptable salts of compounds. Pharmaceutically acceptable salts retain the desired biological activity of the parent compound and do not impart undesired toxicological effects thereto.

As used herein, “potassium salt” means a salt of a modified oligonucleotide, wherein the cation of the salt is potassium.

As used herein, “RNA” means an RNA transcript and includes pre-mRNA and mature mRNA unless otherwise specified.

As used herein, “sodium salt” means a salt of a modified oligonucleotide, wherein the cation of the salt is sodium.

As used herein, “subject” means a human or non-human animal. In certain embodiments, the subject is a human subject. A “subject in need thereof,” is a subject who would benefit from administration of a modified oligonucleotide disclosed herein. In certain embodiments, the subject in need thereof has FCS.

As used herein, “sugar moiety” means an unmodified sugar moiety or a modified sugar moiety. “Unmodified sugar moiety” means a 2′-OH(H) β-D ribosyl moiety, as found in RNA (an “unmodified RNA sugar moiety”), or a 2′-H(H) β-D deoxyribosyl moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. “Modified sugar moiety” or “modified sugar” means a modified furanosyl sugar moiety or a sugar surrogate.

As used herein, “symptom” means any physical feature or test result that indicates the existence or extent of a disease or disorder. In certain embodiments, a symptom is apparent to a subject or to a medical professional examining or testing the subject.

As used herein, “therapeutically effective amount” means an amount of a pharmaceutical agent that provides a therapeutic benefit to a human subject. For example, a therapeutically effective amount improves a symptom of a disease.

As used herein, “week” means 7 days.

### CERTAIN EMBODIMENTS

Embodiment 1. A method of ameliorating Familial Chylomicronemia Syndrome (FCS) in a human subject in need thereof, the method comprising administering to the human subject a therapeutically effective amount of a modified oligonucleotide according to the following chemical structure:

(SEQ ID NO: 3), or a salt thereof.

Embodiment 2. The modified oligonucleotide of embodiment 1, which is the sodium salt or the potassium salt.

Embodiment 3. A method of ameliorating FCS in a human subject in need thereof, the method comprising administering to the human subject a therapeutically effective amount of a modified oligonucleotide according to the following chemical structure:

(SEQ ID NO: 3).

Embodiment 4. A method of ameliorating FCS in a human subject in need thereof, the method comprising administering to the human subject a therapeutically effective amount of a modified oligonucleotide, wherein the modified oligonucleotide has the following chemical notation (5′ to 3′): AₑₛGₑₛ^{m}CₑₛTₑₛTₑₛ^{m}C_{ds}T_{ds}T_{ds}G_{ds}T_{ds}^{m}C_{ds}^{m}C_{ds}A_{ds}G_{ds}^{m}C_{ds}TₑₛTₑₛTₑₛAₑₛTₑ (SEQ ID NO: 3); wherein,
A=an adenine nucleobase,
mC=a 5-methyl cytosine nucleobase,
G=a guanine nucleobase,
T=a thymine nucleobase,
e=a 2′-MOE sugar moiety,
d=a 2′-β-D-deoxyribosyl sugar moiety, and
s=a phosphorothioate internucleoside linkage,

and wherein the modified oligonucleotide has a 5′-trishexylamino-(THA)-C₆GalNAc₃ endcap, represented by the structure below, wherein the phosphate group is attached to the 5′-oxygen atom of the 5′-nucleoside:

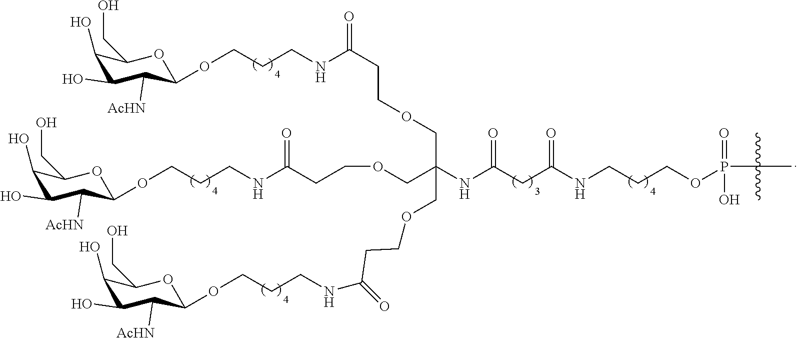

Embodiment 5. The method of any one of embodiments 1-4, wherein at least one symptom of FCS is ameliorated.

Embodiment 6. The method of embodiment 5, wherein the at least one symptom comprises severe chylomicronemia, severe hypertriglyceridemia (always reaching well above 1000 mg/dL and not infrequently rising as high as 10,000 mg/dL or more), frequent and severe abdominal pain, repetitive colicky pain, physical fatigue, difficulty thinking, diarrhea, recurrent acute pancreatitis, eruptive xanthomas, lipemia retinalis, and hepatosplenomegaly or a combination thereof.

Embodiment 7. A method of reducing APOCIII RNA in a human subject in need thereof, the method comprising administering to the human subject a therapeutically effective amount of a modified oligonucleotide according to the following chemical structure:

(SEQ ID NO: 3), or a salt thereof.

Embodiment 8. The modified oligonucleotide of embodiment 7, which is the sodium salt or the potassium salt.

Embodiment 9. A method of reducing APOCIII RNA in a human subject in need thereof, the method comprising administering to the human subject a therapeutically effective amount of a modified oligonucleotide according to the following chemical structure:

(SEQ ID NO: 3).

Embodiment 10. A method of reducing APOCIII RNA in a human subject in need thereof, the method comprising administering to the human subject a therapeutically effective amount of a modified oligonucleotide, wherein the modified oligonucleotide has the following chemical notation (5′ to 3′): AₑₛGₑₛ^{m}CₑₛTₑₛTₑₛ^{m}C_{ds}T_{ds}T_{ds}G_{ds}T_{ds}^{m}C_{ds}^{m}C_{ds}A_{ds}G_{ds}^{m}C_{ds}TₑₛTₑₛTₑₛAₑₛTₑ (SEQ ID NO: 3); wherein,
A=an adenine nucleobase,
mC=a 5-methyl cytosine nucleobase,
G=a guanine nucleobase,
T=a thymine nucleobase,
e=a 2′-MOE sugar moiety,
d=a 2′-β-D-deoxyribosyl sugar moiety, and
s=a phosphorothioate internucleoside linkage,

and wherein the modified oligonucleotide has a 5′-trishexylamino-(THA)-C₆GalNAc₃ endcap, represented by the structure below, wherein the phosphate group is attached to the 5′-oxygen atom of the 5′-nucleoside:

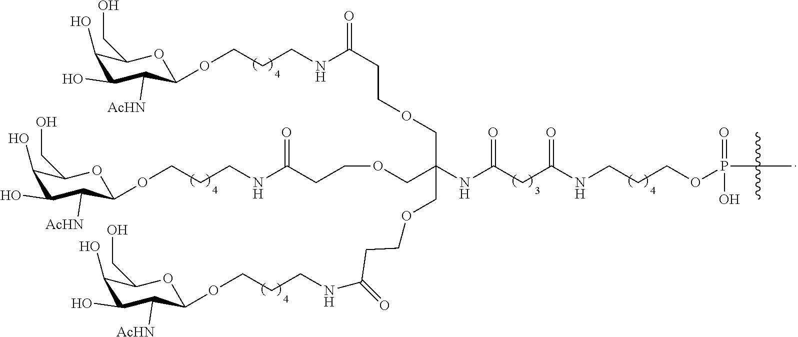

Embodiment 11. The method of any one of embodiments 1-10, wherein the therapeutically effective amount is 50 mg.

Embodiment 12. The method of any one of embodiments 1-10, wherein the therapeutically effective amount is 60 mg.

Embodiment 13. The method of any one of embodiments 1-10, wherein the therapeutically effective amount is 70 mg.

Embodiment 14. The method of any one of embodiments 1-10, wherein the therapeutically effective amount is 80 mg.

Embodiment 15. The method of any one of embodiments 1-10, wherein the therapeutically effective amount is any of 40 mg, 45 mg, 50 mg, 55 mg, 60 mg, 65 mg, 70 mg, 75 mg, 80 mg, 85 mg, 90 mg, 95 mg, and 100 mg.

Embodiment 16. The method of any one of embodiments 1-10, wherein the therapeutically effective amount is any of about 40 mg, about 45 mg, about 50 mg, about 55 mg, about 60 mg, about 65 mg, about 70 mg, about 75 mg, about 80 mg, about 85 mg, about 90 mg, about 95 mg, and about 100 mg.

Embodiment 17. The method of any one of embodiments 1-10, wherein the therapeutically effective amount is any of 40.0 mg, 40.1 mg, 40.2 mg, 40.3 mg, 40.4 mg, 40.5 mg, 40.6 mg, 40.7 mg, 40.8 mg, 40.9 mg, 41.0 mg, 41.1 mg, 41.2 mg, 41.3 mg, 41.4 mg, 41.5 mg, 41.6 mg, 41.7 mg, 41.8 mg, 41.9 mg, 42.0 mg, 42.1 mg, 42.2 mg, 42.3 mg, 42.4 mg, 42.5 mg, 42.6 mg, 42.7 mg, 42.8 mg, 42.9 mg, 43.0 mg, 43.1 mg, 43.2 mg, 43.3 mg, 43.4 mg, 43.5 mg, 43.6 mg, 43.7 mg, 43.8 mg, 43.9 mg, 44.0 mg, 44.1 mg, 44.2 mg, 44.3 mg, 44.4 mg, 44.5 mg, 44.6 mg, 44.7 mg, 44.8 mg, 44.9 mg, 45.0 mg, 45.1 mg, 45.2 mg, 45.3 mg, 45.4 mg, 45.5 mg, 45.6 mg, 45.7 mg, 45.8 mg, 45.9 mg, 46.0 mg, 46.1 mg, 46.2 mg, 46.3 mg, 46.4 mg, 46.5 mg, 46.6 mg, 46.7 mg, 46.8 mg, 46.9 mg, 47.0 mg, 47.1 mg, 47.2 mg, 47.3 mg, 47.4 mg, 47.5 mg, 47.6 mg, 47.7 mg, 47.8 mg, 47.9 mg, 48.0 mg, 48.1 mg, 48.2 mg, 48.3 mg, 48.4 mg, 48.5 mg, 48.6 mg, 48.7 mg, 48.8 mg, 48.9 mg, 49.0 mg, 49.1 mg, 49.2 mg, 49.3 mg, 49.4 mg, 49.5 mg, 49.6 mg, 49.7 mg, 49.8 mg, 49.9 mg, 50.0 mg, 50.1 mg, 50.2 mg, 50.3 mg, 50.4 mg, 50.5 mg, 50.6 mg, 50.7 mg, 50.8 mg, 50.9 mg, 51.0 mg, 51.1 mg, 51.2 mg, 51.3 mg, 51.4 mg, 51.5 mg, 51.6 mg, 51.7 mg, 51.8 mg, 51.9 mg, 52.0 mg, 52.1 mg, 52.2 mg, 52.3 mg, 52.4 mg, 52.5 mg, 52.6 mg, 52.7 mg, 52.8 mg, 52.9 mg, 53.0 mg, 53.1 mg, 53.2 mg, 53.3 mg. 53.4 mg, 53.5 mg, 53.6 mg, 53.7 mg, 53.8 mg, 53.9 mg, 54.0 mg, 54.1 mg, 54.2 mg, 54.3 mg, 54.4 mg, 54.5 mg, 54.6 mg, 54.7 mg, 54.8 mg, 54.9 mg, 55.0 mg, 50.0 mg, 50.1 mg, 50.2 mg, 50.3 mg, 50.4 mg, 50.5 mg, 50.6 mg, 50.7 mg, 50.8 mg, 50.9 mg, 51.0 mg, 51.1 mg, 51.2 mg, 51.3 mg, 51.4 mg, 51.5 mg, 51.6 mg, 51.7 mg, 51.8 mg, 51.9 mg, 52.0 mg, 52.1 mg, 52.2 mg, 52.3 mg, 52.4 mg, 52.5 mg, 52.6 mg, 52.7 mg, 52.8 mg, 52.9 mg, 53.0 mg, 53.1 mg, 53.2 mg, 53.3 mg. 53.4 mg, 53.5 mg, 53.6 mg, 53.7 mg, 53.8 mg, 53.9 mg, 54.0 mg, 54.1 mg, 54.2 mg, 54.3 mg, 54.4 mg, 54.5 mg, 54.6 mg, 54.7 mg, 54.8 mg, 54.9 mg, 55.0 mg, 55.1 mg, 55.2 mg, 55.3 mg. 55.4 mg, 55.5 mg, 55.6 mg, 55.7 mg, 55.8 mg, 55.9 mg, 55.0 mg, 55.1 mg, 55.2 mg, 55.3 mg, 55.4 mg, 55.5 mg, 55.6 mg, 55.7 mg, 55.8 mg, 55.9 mg, 56.0 mg, 56.1 mg, 56.2 mg, 56.3 mg. 56.4 mg, 56.5 mg, 56.6 mg, 56.7 mg, 56.8 mg, 56.9 mg, 57.0 mg, 57.1 mg, 57.2 mg, 57.3 mg, 57.4 mg, 57.5 mg, 57.6 mg, 57.7 mg, 57.8 mg, 57.9 mg, 58.0 mg, 58.1 mg, 58.2 mg, 58.3 mg. 58.4 mg, 58.5 mg, 58.6 mg, 58.7 mg, 58.8 mg, 58.9 mg, 59.0 mg, 59.1 mg, 59.2 mg, 59.3 mg, 59.4 mg, 59.5 mg, 59.6 mg, 59.7 mg, 59.8 mg, 59.9 mg, 60.0 mg, 60.1 mg, 60.2 mg, 60.3 mg, 60.4 mg, 60.5 mg, 60.6 mg, 60.7 mg, 60.8 mg, 60.9 mg, 61.0 mg, 61.1 mg, 61.2 mg, 61.3 mg, 61.4 mg, 61.5 mg, 61.6 mg, 61.7 mg, 61.8 mg, 61.9 mg, 62.0 mg, 62.1 mg, 62.2 mg, 62.3 mg, 62.4 mg, 62.5 mg, 62.6 mg, 62.7 mg, 62.8 mg, 62.9 mg, 63.0 mg, 63.1 mg, 63.2 mg, 63.3 mg, 63.4 mg, 63.5 mg, 63.6 mg, 63.7 mg, 63.8 mg, 63.9 mg, 64.0 mg, 64.1 mg, 64.2 mg, 64.3 mg, 64.4 mg, 64.5 mg, 64.6 mg, 64.7 mg, 64.8 mg, 64.9 mg, 65.0 mg, 65.1 mg, 65.2 mg, 65.3 mg, 65.4 mg, 65.5 mg, 65.6 mg, 65.7 mg, 65.8 mg, 65.9 mg, 66.0 mg, 66.1 mg, 66.2 mg, 66.3 mg, 66.4 mg, 66.5 mg, 66.6 mg, 66.7 mg, 66.8 mg, 66.9 mg, 67.0 mg, 67.1 mg, 67.2 mg, 67.3 mg, 67.4 mg, 67.5 mg, 67.6 mg, 67.7 mg, 67.8 mg, 67.9 mg, 68.0 mg, 68.1 mg, 68.2 mg, 68.3 mg, 68.4 mg, 68.5 mg, 68.6 mg, 68.7 mg, 68.8 mg, 68.9 mg, 69.0 mg, 69.1 mg, 69.2 mg, 69.3 mg, 69.4 mg, 69.5 mg, 69.6 mg, 69.7 mg, 69.8 mg, 69.9 mg, 70.0 mg, 70.1 mg, 70.2 mg, 70.3 mg, 70.4 mg, 70.5 mg, 70.6 mg, 70.7 mg, 70.8 mg, 70.9 mg, 71.0 mg, 71.1 mg, 71.2 mg, 71.3 mg, 71.4 mg, 71.5 mg, 71.6 mg, 71.7 mg, 71.8 mg, 71.9 mg, 72.0 mg, 72.1 mg, 72.2 mg, 72.3 mg, 72.4 mg, 72.5 mg, 72.6 mg, 72.7 mg, 72.8 mg, 72.9 mg, 73.0 mg, 73.1 mg, 73.2 mg, 73.3 mg, 73.4 mg, 73.5 mg, 73.6 mg, 73.7 mg, 73.8 mg, 73.9 mg, 74.0 mg, 74.1 mg, 74.2 mg, 74.3 mg, 74.4 mg, 74.5 mg, 74.6 mg, 74.7 mg, 74.8 mg, 74.9 mg, 75.0 mg, 75.1 mg, 75.2 mg, 75.3 mg, 75.4 mg, 75.5 mg, 75.6 mg, 75.7 mg, 75.8 mg, 75.9 mg, 76.0 mg, 76.1 mg, 76.2 mg, 76.3 mg, 76.4 mg, 76.5 mg, 76.6 mg, 76.7 mg, 76.8 mg, 76.9 mg, 77.0 mg, 77.1 mg, 77.2 mg, 77.3 mg, 77.4 mg, 77.5 mg, 77.6 mg, 77.7 mg, 77.8 mg, 77.9 mg, 78.0 mg, 78.1 mg, 78.2 mg, 78.3 mg, 78.4 mg, 78.5 mg, 78.6 mg, 78.7 mg, 78.8 mg, 78.9 mg, 79.0 mg, 79.1 mg, 79.2 mg, 79.3 mg, 79.4 mg, 79.5 mg, 79.6 mg, 79.7 mg, 79.8 mg, 79.9 mg, 80.0 mg, 80.1 mg, 80.2 mg, 80.3 mg, 80.4 mg, 80.5 mg, 80.6 mg, 80.7 mg, 80.8 mg, 80.9 mg, 81.0 mg, 81.1 mg, 81.2 mg, 81.3 mg, 81.4 mg, 81.5 mg, 81.6 mg, 81.7 mg, 81.8 mg, 81.9 mg, 82.0 mg, 82.0 mg, 82.1 mg, 82.2 mg, 82.3 mg, 82.4 mg, 82.5 mg, 82.6 mg, 82.7 mg, 82.8 mg, 82.9 mg, 83.0 mg, 83.1 mg, 83.2 mg, 83.3 mg, 83.4 mg, 83.5 mg, 83.6 mg, 83.7 mg, 83.8 mg, 83.9 mg, 84.0 mg, 84.1 mg, 84.2 mg, 84.3 mg, 84.4 mg, 84.5 mg, 84.6 mg, 84.7 mg, 84.8 mg, 84.9 mg, 85.0 mg, 85.0 mg, 85.1 mg, 85.2 mg, 85.3 mg, 85.4 mg, 85.5 mg, 85.6 mg, 85.7 mg, 85.8 mg, 85.9 mg, 86.0 mg, 86.1 mg, 86.2 mg, 86.3 mg, 86.4 mg, 86.5 mg, 86.6 mg, 86.7 mg, 86.8 mg, 86.9 mg, 87.0 mg, 87.1 mg, 87.2 mg, 87.3 mg, 87.4 mg, 87.5 mg, 87.6 mg, 87.7 mg, 87.8 mg, 87.9 mg, 88.0 mg, 88.1 mg, 88.2 mg, 88.3 mg, 88.4 mg, 88.5 mg, 88.6 mg, 88.7 mg, 88.8 mg, 88.9 mg, 89.0 mg, 89.0 mg, 89.1 mg, 89.2 mg, 89.3 mg, 89.4 mg, 89.5 mg, 89.6 mg, 89.7 mg, 89.8 mg, 89.9 mg, 90.0 mg, 90.1 mg, 90.2 mg, 90.3 mg, 90.4 mg, 90.5 mg, 90.6 mg, 90.7 mg, 90.8 mg, 90.9 mg, 91.0 mg, 91.1 mg, 91.2 mg, 91.3 mg, 91.4 mg, 91.5 mg, 91.6 mg, 91.7 mg, 91.8 mg, 91.9 mg, 92.0 mg, 92.0 mg, 92.1 mg, 92.2 mg, 92.3 mg, 92.4 mg, 92.5 mg, 92.6 mg, 92.7 mg, 92.8 mg, 92.9 mg, 93.0 mg, 93.1 mg, 93.2 mg, 93.3 mg, 93.4 mg, 93.5 mg, 93.6 mg, 93.7 mg, 93.8 mg, 93.9 mg, 94.0 mg, 94.1 mg, 94.2 mg, 94.3 mg, 94.4 mg, 94.5 mg, 94.6 mg, 94.7 mg, 94.8 mg, 94.9 mg, 95.0 mg, 95.1 mg, 95.2 mg, 95.3 mg, 95.4 mg, 95.5 mg, 95.6 mg, 95.7 mg, 95.8 mg, 95.9 mg, 96.0 mg, 96.1 mg, 96.2 mg, 96.3 mg, 96.4 mg, 96.5 mg, 96.6 mg, 96.7 mg, 96.8 mg, 96.9 mg, 97.0 mg, 97.1 mg, 97.2 mg, 97.3 mg, 97.4 mg, 97.5 mg, 97.6 mg, 97.7 mg, 97.8 mg, 97.9 mg, 98.0 mg, 98.1 mg, 98.2 mg, 98.3 mg, 98.4 mg, 98.5 mg, 98.6 mg, 98.7 mg, 98.8 mg, 98.9 mg, 99.0 mg, 99.1 mg, 99.2 mg, 99.3 mg, 99.4 mg, 99.5 mg, 99.6 mg, 99.7 mg, 99.8 mg, 99.9 mg, and 100.0 mg.

Embodiment 18. The method of any one of embodiments 1-10, wherein the therapeutically effective amount is any of about 40.0 mg, about 40.1 mg, about 40.2 mg, about 40.3 mg, about 40.4 mg, about 40.5 mg, about 40.6 mg, about 40.7 mg, about 40.8 mg, about 40.9 mg, about 41.0 mg, about 41.1 mg, about 41.2 mg, about 41.3 mg, about 41.4 mg, about 41.5 mg, about 41.6 mg, about 41.7 mg, about 41.8 mg, about 41.9 mg, about 42.0 mg, about 42.1 mg, about 42.2 mg, about 42.3 mg, about 42.4 mg, about 42.5 mg, about 42.6 mg, about 42.7 mg, about 42.8 mg, about 42.9 mg, about 43.0 mg, about 43.1 mg, about 43.2 mg, about 43.3 mg, about 43.4 mg, about 43.5 mg, about 43.6 mg, about 43.7 mg, about 43.8 mg, about 43.9 mg, about 44.0 mg, about 44.1 mg, about 44.2 mg, about 44.3 mg, about 44.4 mg, about 44.5 mg, about 44.6 mg, about 44.7 mg, about 44.8 mg, about 44.9 mg, about 45.0 mg, about 45.1 mg, about 45.2 mg, about 45.3 mg, about 45.4 mg, about 45.5 mg, about 45.6 mg, about 45.7 mg, about 45.8 mg, about 45.9 mg, about 46.0 mg, about 46.1 mg, about 46.2 mg, about 46.3 mg, about 46.4 mg, about 46.5 mg, about 46.6 mg, about 46.7 mg, about 46.8 mg, about 46.9 mg, about 47.0 mg, about 47.1 mg, about 47.2 mg, about 47.3 mg, about 47.4 mg, about 47.5 mg, about 47.6 mg, about 47.7 mg, about 47.8 mg, about 47.9 mg, about 48.0 mg, about 48.1 mg, about 48.2 mg, about 48.3 mg, about 48.4 mg, about 48.5 mg, about 48.6 mg, about 48.7 mg, about 48.8 mg, about 48.9 mg, about 49.0 mg, about 49.1 mg, about 49.2 mg, about 49.3 mg, about 49.4 mg, about 49.5 mg, about 49.6 mg, about 49.7 mg, about 49.8 mg, about 49.9 mg, about 50.0 mg, about 50.1 mg, about 50.2 mg, about 50.3 mg, about 50.4 mg, about 50.5 mg, about 50.6 mg, about 50.7 mg, about 50.8 mg, about 50.9 mg, about 51.0 mg, about 51.1 mg, about 51.2 mg, about 51.3 mg, about 51.4 mg, about 51.5 mg, about 51.6 mg, about 51.7 mg, about 51.8 mg, about 51.9 mg, about 52.0 mg, about 52.1 mg, about 52.2 mg, about 52.3 mg, about 52.4 mg, about 52.5 mg, about 52.6 mg, about 52.7 mg, about 52.8 mg, about 52.9 mg, about 53.0 mg, about 53.1 mg, about 53.2 mg, about 53.3 mg. about 53.4 mg, about 53.5 mg, about 53.6 mg, about 53.7 mg, about 53.8 mg, about 53.9 mg, about 54.0 mg, about 54.1 mg, about 54.2 mg, about 54.3 mg, about 54.4 mg, about 54.5 mg, about 54.6 mg, about 54.7 mg, about 54.8 mg, about 54.9 mg, about 55.0 mg, about 55.1 mg, about 55.2 mg, about 55.3 mg. about 55.4 mg, about 55.5 mg, about 55.6 mg, about 55.7 mg, about 55.8 mg, about 55.9 mg, about 55.0 mg, about 55.1 mg, about 55.2 mg, about 55.3 mg, about 55.4 mg, about 55.5 mg, about 55.6 mg, about 55.7 mg, about 55.8 mg, about 55.9 mg, about 56.0 mg, about 56.1 mg, about 56.2 mg, about 56.3 mg. about 56.4 mg, about 56.5 mg, about 56.6 mg, about 56.7 mg, about 56.8 mg, about 56.9 mg, about 57.0 mg, about 57.1 mg, about 57.2 mg, about 57.3 mg, about 57.4 mg, about 57.5 mg, about 57.6 mg, about 57.7 mg, about 57.8 mg, about 57.9 mg, about 58.0 mg, about 58.1 mg, about 58.2 mg, about 58.3 mg. about 58.4 mg, about 58.5 mg, about 58.6 mg, about 58.7 mg, about 58.8 mg, about 58.9 mg, about 59.0 mg, about 59.1 mg, about 59.2 mg, about 59.3 mg, about 59.4 mg, about 59.5 mg, about 59.6 mg, about 59.7 mg, about 59.8 mg, about 59.9 mg, about 60.0 mg, about 60.1 mg, about 60.2 mg, about 60.3 mg, about 60.4 mg, about 60.5 mg, about 60.6 mg, about 60.7 mg, about 60.8 mg, about 60.9 mg, about 61.0 mg, about 61.1 mg, about 61.2 mg, about 61.3 mg, about 61.4 mg, about 61.5 mg, about 61.6 mg, about 61.7 mg, about 61.8 mg, about 61.9 mg, about 62.0 mg, about 62.1 mg, about 62.2 mg, about 62.3 mg, about 62.4 mg, about 62.5 mg, about 62.6 mg, about 62.7 mg, about 62.8 mg, about 62.9 mg, about 63.0 mg, about 63.1 mg, about 63.2 mg, about 63.3 mg, about 63.4 mg, about 63.5 mg, about 63.6 mg, about 63.7 mg, about 63.8 mg, about 63.9 mg, about 64.0 mg, about 64.1 mg, about 64.2 mg, about 64.3 mg, about 64.4 mg, about 64.5 mg, about 64.6 mg, about 64.7 mg, about 64.8 mg, about 64.9 mg, about 65.0 mg, about 65.1 mg, about 65.2 mg, about 65.3 mg, about 65.4 mg, about 65.5 mg, about 65.6 mg, about 65.7 mg, about 65.8 mg, about 65.9 mg, about 66.0 mg, about 66.1 mg, about 66.2 mg, about 66.3 mg, about 66.4 mg, about 66.5 mg, about 66.6 mg, about 66.7 mg, about 66.8 mg, about 66.9 mg, about 67.0 mg, about 67.1 mg, about 67.2 mg, about 67.3 mg, about 67.4 mg, about 67.5 mg, about 67.6 mg, about 67.7 mg, about 67.8 mg, about 67.9 mg, about 68.0 mg, about 68.1 mg, about 68.2 mg, about 68.3 mg, about 68.4 mg, about 68.5 mg, about 68.6 mg, about 68.7 mg, about 68.8 mg, about 68.9 mg, about 69.0 mg, about 69.1 mg, about 69.2 mg, about 69.3 mg, about 69.4 mg, about 69.5 mg, about 69.6 mg, about 69.7 mg, about 69.8 mg, about 69.9 mg, about 70.0 mg, about 70.1 mg, about 70.2 mg, about 70.3 mg, about 70.4 mg, about 70.5 mg, about 70.6 mg, about 70.7 mg, about 70.8 mg, about 70.9 mg, about 71.0 mg, about 71.1 mg, about 71.2 mg, about 71.3 mg, about 71.4 mg, about 71.5 mg, about 71.6 mg, about 71.7 mg, about 71.8 mg, about 71.9 mg, about 72.0 mg, about 72.1 mg, about 72.2 mg, about 72.3 mg, about 72.4 mg, about 72.5 mg, about 72.6 mg, about 72.7 mg, about 72.8 mg, about 72.9 mg, about 73.0 mg, about 73.1 mg, about 73.2 mg, about 73.3 mg, about 73.4 mg, about 73.5 mg, about 73.6 mg, about 73.7 mg, about 73.8 mg, about 73.9 mg, about 74.0 mg, about 74.1 mg, about 74.2 mg, about 74.3 mg, about 74.4 mg, about 74.5 mg, about 74.6 mg, about 74.7 mg, about 74.8 mg, about 74.9 mg, about 75.0 mg, about 75.1 mg, about 75.2 mg, about 75.3 mg, about 75.4 mg, about 75.5 mg, about 75.6 mg, about 75.7 mg, about 75.8 mg, about 75.9 mg, about 76.0 mg, about 76.1 mg, about 76.2 mg, about 76.3 mg, about 76.4 mg, about 76.5 mg, about 76.6 mg, about 76.7 mg, about 76.8 mg, about 76.9 mg, about 77.0 mg, about 77.1 mg, about 77.2 mg, about 77.3 mg, about 77.4 mg, about 77.5 mg, about 77.6 mg, about 77.7 mg, about 77.8 mg, about 77.9 mg, about 78.0 mg, about 78.1 mg, about 78.2 mg, about 78.3 mg, about 78.4 mg, about 78.5 mg, about 78.6 mg, about 78.7 mg, about 78.8 mg, about 78.9 mg, about 79.0 mg, about 79.1 mg, about 79.2 mg, about 79.3 mg, about 79.4 mg, about 79.5 mg, about 79.6 mg, about 79.7 mg, about 79.8 mg, about 79.9 mg, about 80.0 mg, about 80.1 mg, about 80.2 mg, about 80.3 mg, about 80.4 mg, about 80.5 mg, about 80.6 mg, about 80.7 mg, about 80.8 mg, about 80.9 mg, about 81.0 mg, about 81.1 mg, about 81.2 mg, about 81.3 mg, about 81.4 mg, about 81.5 mg, about 81.6 mg, about 81.7 mg, about 81.8 mg, about 81.9 mg, about 82.0 mg, about 82.0 mg, about 82.1 mg, about 82.2 mg, about 82.3 mg, about 82.4 mg, about 82.5 mg, about 82.6 mg, about 82.7 mg, about 82.8 mg, about 82.9 mg, about 83.0 mg, about 83.1 mg, about 83.2 mg, about 83.3 mg, about 83.4 mg, about 83.5 mg, about 83.6 mg, about 83.7 mg, about 83.8 mg, about 83.9 mg, about 84.0 mg, about 84.1 mg, about 84.2 mg, about 84.3 mg, about 84.4 mg, about 84.5 mg, about 84.6 mg, about 84.7 mg, about 84.8 mg, about 84.9 mg, about 85.0 mg, about 85.1 mg, about 85.2 mg, about 85.3 mg, about 85.4 mg, about 85.5 mg, about 85.6 mg, about 85.7 mg, about 85.8 mg, about 85.9 mg, about 86.0 mg, about 86.1 mg, about 86.2 mg, about 86.3 mg, about 86.4 mg, about 86.5 mg, about 86.6 mg, about 86.7 mg, about 86.8 mg, about 86.9 mg, about 87.0 mg, about 87.1 mg, about 87.2 mg, about 87.3 mg, about 87.4 mg, about 87.5 mg, about 87.6 mg, about 87.7 mg, about 87.8 mg, about 87.9 mg, about 88.0 mg, about 88.1 mg, about 88.2 mg, about 88.3 mg, about 88.4 mg, about 88.5 mg, about 88.6 mg, about 88.7 mg, about 88.8 mg, about 88.9 mg, about 89.0 mg, about 89.1 mg, about 89.2 mg, about 89.3 mg, about 89.4 mg, about 89.5 mg, about 89.6 mg, about 89.7 mg, about 89.8 mg, about 89.9 mg, about 90.0 mg, about 90.1 mg, about 90.2 mg, about 90.3 mg, about 90.4 mg, about 90.5 mg, about 90.6 mg, about 90.7 mg, about 90.8 mg, about 90.9 mg, about 91.0 mg, about 91.1 mg, about 91.2 mg, about 91.3 mg, about 91.4 mg, about 91.5 mg, about 91.6 mg, about 91.7 mg, about 91.8 mg, about 91.9 mg, about 92.0 mg, about 92.1 mg, about 92.2 mg, about 92.3 mg, about 92.4 mg, about 92.5 mg, about 92.6 mg, about 92.7 mg, about 92.8 mg, about 92.9 mg, about 93.0 mg, about 93.1 mg, about 93.2 mg, about 93.3 mg, about 93.4 mg, about 93.5 mg, about 93.6 mg, about 93.7 mg, about 93.8 mg, about 93.9 mg, about 94.0 mg, about 94.1 mg, about 94.2 mg, about 94.3 mg, about 94.4 mg, about 94.5 mg, about 94.6 mg, about 94.7 mg, about 94.8 mg, about 94.9 mg, about 95.0 mg, about 95.1 mg, about 95.2 mg, about 95.3 mg, about 95.4 mg, about 95.5 mg, about 95.6 mg, about 95.7 mg, about 95.8 mg, about 95.9 mg, about 96.0 mg, about 96.1 mg, about 96.2 mg, about 96.3 mg, about 96.4 mg, about 96.5 mg, about 96.6 mg, about 96.7 mg, about 96.8 mg, about 96.9 mg, about 97.0 mg, about 97.1 mg, about 97.2 mg, about 97.3 mg, about 97.4 mg, about 97.5 mg, about 97.6 mg, about 97.7 mg, about 97.8 mg, about 97.9 mg, about 98.0 mg, about 98.1 mg, about 98.2 mg, about 98.3 mg, about 98.4 mg, about 98.5 mg, about 98.6 mg, about 98.7 mg, about 98.8 mg, about 98.9 mg, about 99.0 mg, about 99.1 mg, about 99.2 mg, about 99.3 mg, about 99.4 mg, about 99.5 mg, about 99.6 mg, about 99.7 mg, about 99.8 mg, about 99.9 mg, and about 100.0 mg.

Embodiment 19. The method of any one of embodiments 1-10, wherein the therapeutically effective amount is within the range of any of 40 mg to 100 mg, 40 mg to 80 mg, 40 mg to 70 mg, 40 mg to 60 mg, 40 mg to 50 mg, 50 mg to 100 mg, 50 mg to 80 mg, 50 mg to 70 mg, 50 mg to 60 mg, 60 mg to 100 mg, 60 mg to 80 mg, 60 mg to 70 mg, 70 mg to 100 mg, 70 mg to 80 mg, 80 mg to 100 mg, 80 mg to 90 mg, and 90 mg to 100 mg.

Embodiment 20. The method of any one of embodiments 1-10, wherein the therapeutically effective amount is any of less than 100 mg, less than 95 mg, less than 90 mg, less than 85 mg, less than 80 mg, less than 75 mg, less than 70 mg, less than 65 mg, less than 60 mg, less than 55 mg, less than 50 mg, less than 45 mg, and less than 40 mg.

Embodiment 21. The method of any one of embodiments 1-10, wherein the therapeutically effective amount is any of less than about less than about 100 mg, less than about 95 mg, less than about 90 mg, less than about 85 mg, less than about 80 mg, less than about 75 mg, less than about 70 mg, less than about 65 mg, less than about 60 mg, less than about 55 mg, less than about 50 mg, less than about 45 mg, and less than about 40 mg.

Embodiment 22. The method of any one of embodiments 1-10, wherein the therapeutically effective amount is any of at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, and at least about 100 mg.

Embodiment 23. The method of any one of embodiments 1-10, wherein the therapeutically effective amount is any of at least about 40 mg, at least about 45 mg, at least about 50 mg, at least about 55 mg, at least about 60 mg, at least about 65 mg, at least about 70 mg, at least about 75 mg, at least about 80 mg, at least about 85 mg, at least about 90 mg, at least about 95 mg, and at least about 100 mg.

Embodiment 24. The method of any one of embodiments 1-23, comprising administering the modified oligonucleotide once every 4 weeks.

Embodiment 25. The method of any one of embodiments 1-23, comprising administering the modified oligonucleotide once every 8 weeks.

Embodiment 26. The method of any one of embodiments 1-23, comprising administering the modified oligonucleotide once every 12 weeks.

Embodiment 27. The method of any one of embodiments 1-23, comprising administering the modified oligonucleotide once every 16 weeks.

Embodiment 28. The method of any one of embodiments 1-23, comprising administering the modified oligonucleotide once every 20 weeks.

Embodiment 29. The method of any one of embodiments 1-23, comprising administering the modified oligonucleotide about once every 4 weeks.

Embodiment 30. The method of any one of embodiments 1-23, comprising administering the modified oligonucleotide about once every 8 weeks.

Embodiment 31. The method of any one of embodiments 1-23, comprising administering the modified oligonucleotide about once every 12 weeks.

Embodiment 32. The method of any one of embodiments 1-23, comprising administering the modified oligonucleotide about once every 16 weeks.

Embodiment 33. The method of any one of embodiments 1-23, comprising administering the modified oligonucleotide about once every 20 weeks.

Embodiment 34. The method of any one of embodiments 1-23, comprising administering the modified oligonucleotide any of once every 1 week, once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks, once every 17 weeks, once every 18 weeks, once every 19 weeks, and once every 20 weeks.

Embodiment 35. The method of any one of embodiments 1-23, comprising administering the modified oligonucleotide any of once about every 1 week, once about every 2 weeks, once about every 3 weeks, once about every 4 weeks, once about every 5 weeks, once about every 6 weeks, once about every 7 weeks, once about every 8 weeks, once about every 9 weeks, once about every 10 weeks, once about every 11 weeks, once about every 12 weeks, once about every 13 weeks, once about every 14 weeks, once about every 15 weeks, once about every 16 weeks, once about every 17 weeks, once about every 18 weeks, once about every 19 weeks, and once about every 20 weeks.

Embodiment 36. A method of ameliorating FCS, reducing APOCIII RNA, or reducing APOCIII protein, in a human subject in need thereof, the method comprising subcutaneously administering to the human subject a therapeutically effective amount of 50 mg or about 50 mg of a modified oligonucleotide according to the following chemical structure:

(SEQ ID NO: 3), or a salt thereof.

Embodiment 37. A method of ameliorating FCS, reducing APOCIII RNA, or reducing APOCIII protein, in a human subject in need thereof, the method comprising subcutaneously administering to the human subject a therapeutically effective amount of 80 mg or about 80 mg of a modified oligonucleotide according to the following chemical structure:

(SEQ ID NO: 3), or a salt thereof.

Embodiment 38. The modified oligonucleotide of embodiment 36 or embodiment 37, which is the sodium salt or the potassium salt.

Embodiment 39. A method of ameliorating FCS, reducing APOCIII RNA, or reducing APOCIII protein, in a human subject in need thereof, the method comprising intrathecally administering to the human subject a therapeutically effective amount of 50 mg or about 50 mg of a modified oligonucleotide according to the following chemical structure:

(SEQ ID NO: 3).

Embodiment 40. A method of ameliorating FCS, reducing APOCIII RNA, or reducing APOCIII protein, in a human subject in need thereof, the method comprising subcutaneously administering to the human subject a therapeutically effective amount of 80 mg or about 80 mg of a modified oligonucleotide according to the following chemical structure:

Embodiment 41. A method of ameliorating FCS, reducing APOCIII RNA, or reducing APOCIII protein in a human subject in need thereof, the method comprising subcutaneously administering to the human subject a therapeutically effective amount of 50 mg or about 50 mg of a modified oligonucleotide, wherein the modified oligonucleotide has the following chemical notation (5′ to 3′): AₑₛGₑₛ^{m}CₑₛTₑₛTₑₛ^{m}C_{ds}T_{ds}T_{ds}G_{ds}T_{ds}^{m}C_{ds}^{m}C_{ds}A_{ds}G_{ds}^{m}C_{ds}TₑₛTₑₛTₑₛAₑₛTₑ (SEQ ID NO: 3); wherein,
A=an adenine nucleobase,
mC=a 5-methyl cytosine nucleobase,
G=a guanine nucleobase,
T=a thymine nucleobase,
e=a 2′-MOE sugar moiety,
d=a 2′-β-D-deoxyribosyl sugar moiety, and
s=a phosphorothioate internucleoside linkage,

and wherein the modified oligonucleotide has a 5′-trishexylamino-(THA)-C₆GalNAc₃ endcap, represented by the structure below, wherein the phosphate group is attached to the 5′-oxygen atom of the 5′-nucleoside:

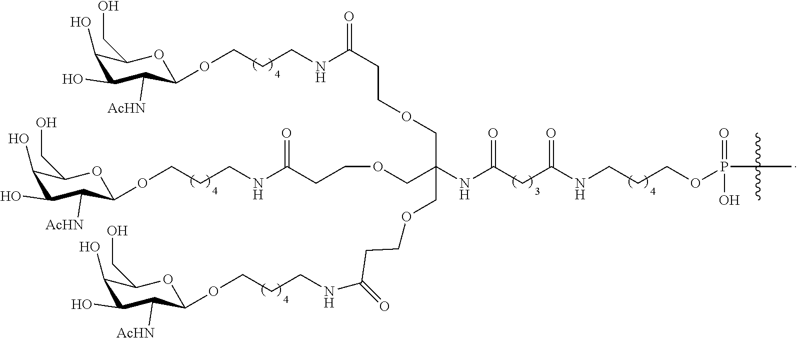

Embodiment 42. A method of ameliorating FCS, reducing APOCIII RNA, or reducing APOCIII protein in a human subject in need thereof, the method comprising subcutaneously administering to the human subject a therapeutically effective amount of 80 mg or about 80 mg of a modified oligonucleotide, wherein the modified oligonucleotide has the following chemical notation (5′ to 3′): AₑₛGₑₛ^{m}CₑₛTₑₛTₑₛ^{m}C_{ds}T_{ds}T_{ds}G_{ds}T_{ds}^{m}C_{ds}^{m}C_{ds}A_{ds}G_{ds}^{m}C_{ds}TₑₛTₑₛTₑₛAₑₛTₑ (SEQ ID NO: 3); wherein,
A=an adenine nucleobase,
mC=a 5-methyl cytosine nucleobase,
G=a guanine nucleobase,
T=a thymine nucleobase,
e=a 2′-MOE sugar moiety,
d=a 2′-β-D-deoxyribosyl sugar moiety, and
s=a phosphorothioate internucleoside linkage,

and wherein the modified oligonucleotide has a 5′-trishexylamino-(THA)-C₆GalNAc₃ endcap, represented by the structure below, wherein the phosphate group is attached to the 5′-oxygen atom of the 5′-nucleoside:

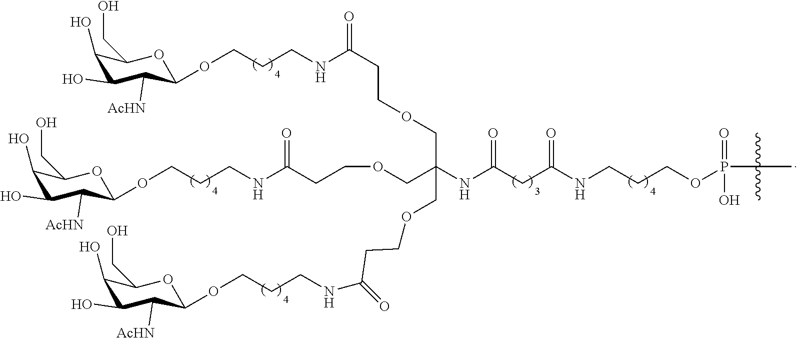

Embodiment 43. The method of any one of embodiments 36-42, comprising administering the modified oligonucleotide about once every 4 weeks.

Embodiment 44. The method of any one of embodiments 36-42, comprising administering the modified oligonucleotide about once every 8 weeks.

Embodiment 45. The method of any one of embodiments 36-42, comprising administering the modified oligonucleotide about once every 16 weeks.

Embodiment 46. The method of any one of embodiments 36-45, wherein at least one symptom of FCS is ameliorated.

Embodiment 47. The method of embodiment 46, wherein the at least one symptom comprises severe chylomicronemia, severe hypertriglyceridemia (always reaching well above 1000 mg/dL and not infrequently rising as high as 10,000 mg/dL or more), frequent and severe abdominal pain, repetitive colicky pain, physical fatigue, difficulty thinking, diarrhea, recurrent acute pancreatitis, eruptive xanthomas, lipemia retinalis, and hepatosplenomegaly, or a combination thereof.

Embodiment 48. The method of any of embodiments 1-47, wherein APOCIII RNA is reduced.

Embodiment 49. The method of any of embodiments 1-47, wherein APOCIII protein is reduced.

Embodiment 50. The method of any one of embodiments 1-49, comprising detecting fasting triglyceride levels in a biological sample from the human subject.

Embodiment 51: The method of any one of embodiments 1-49, comprising detecting fasting apoB-48 levels in a biological sample from the human subject.

Embodiment 52. The method of any of embodiments 50 and 51, wherein the biological sample is plasma.

Embodiment 53: The method of any one of embodiments 1-49, comprising recording the adjudicated acute pancreatitis event rate of the human subject.

Embodiment 54. The method of any of embodiments 50-52, wherein the detecting occurs before the administering.

Embodiment 55. The method of any of embodiments 50-52, wherein the detecting occurs after the administering.

Embodiment 56. The method of any of embodiments 50-52, wherein the detecting occurs before and after the administering.

Embodiment 57. The method of any one of embodiments 50-56, comprising adjusting the initial therapeutically effective amount administered after detecting the amount of APOCIII RNA, or APOCIII protein, or combination thereof.

Embodiment 58. The method of embodiment 57, wherein the dose is at least about 10%, at least about 20%, at least about 30%, at least about 40%, at least about 50%, at least about 60%, at least about 70%, at least about 90%, or at least about 100% greater than the therapeutically effective amount.

Embodiment 59. The method of any one of embodiments 50-58, wherein the therapeutically effective amount is about 50 mg or 80 mg.

Embodiment 60. A method of ameliorating Familial Chylomicronemia Syndrome (FCS), severe hypertriglyceridemia (SHTG), or Familial Partial Lipodystrophy (FPL) in a human subject in need thereof, the method comprising administering to the human subject a therapeutically effective amount of a compound according to the following chemical structure:

(SEQ ID NO: 3), or a salt thereof.

Embodiment 61. The compound of embodiment 1, which is the sodium salt or the potassium salt.

Embodiment 62. A method of ameliorating Familial Chylomicronemia Syndrome (FCS), severe hypertriglyceridemia (SHTG), or Familial Partial Lipodystrophy (FPL) in a human subject in need thereof, the method comprising administering to the human subject a therapeutically effective amount of a compound according to the following chemical structure:

(SEQ ID NO: 3).

Embodiment 63. A method of ameliorating Familial Chylomicronemia Syndrome (FCS), severe hypertriglyceridemia (SHTG), or Familial Partial Lipodystrophy (FPL) in a human subject in need thereof, the method comprising administering to the human subject a therapeutically effective amount of a compound comprising a modified oligonucleotide having the following chemical notation (5′ to 3′): AₑₛGₑₛ^{m}CₑₛTₑₛTₑₛ^{m}C_{ds}T_{ds}T_{ds}G_{ds}T_{ds}^{m}C_{ds}^{m}C_{ds}A_{ds}G_{ds}^{m}C_{ds}TₑₛTₑₛTₑₛAₑₛTₑ (SEQ ID NO: 3); wherein,
A=an adenine nucleobase,
mC=a 5-methyl cytosine nucleobase,
G=a guanine nucleobase,
T=a thymine nucleobase,
e=a 2′-OCH₂CH₂OCH₃ modified ribosyl sugar moiety,
d=a 2′-β-D-deoxyribosyl sugar moiety, and
s=a phosphorothioate internucleoside linkage,

and wherein the modified oligonucleotide has a 5′-trishexylamino-(THA)-C₆GalNAc₃ endcap, represented by the structure below, wherein the phosphate group is attached to the 5′-oxygen atom of the 5′-nucleoside:

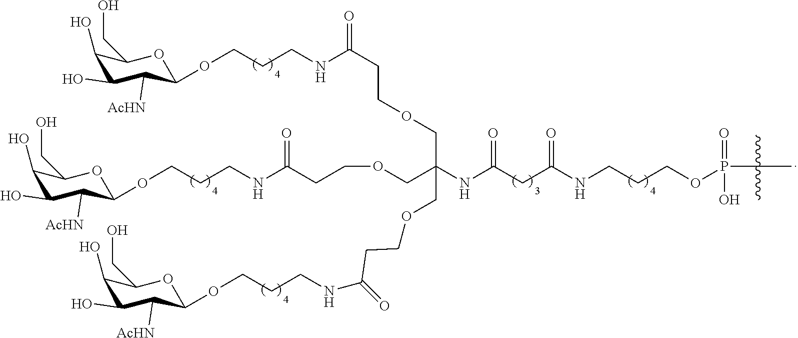

Embodiment 64. The method of any one of embodiments 60-63, wherein at least one symptom of Familial Chylomicronemia Syndrome (FCS), severe hypertriglyceridemia (SHTG), or Familial Partial Lipodystrophy (FPL) is ameliorated.

Embodiment 65. The method of embodiment 64, wherein the at least one symptom of FCS comprises chylomicronemia, hypertriglyceridemia of at least 1000 mg/dL triglycerides, abdominal pain, colicky pain, physical fatigue, difficulty thinking, diarrhea, acute pancreatitis, eruptive xanthomas, lipemia retinalis, and hepatosplenomegaly, or a combination thereof.

Embodiment 66. The method of embodiment 64, wherein the at least one symptom of SHTG comprises chylomicronemia, abdominal pain, colicky pain, physical fatigue, difficulty thinking, diarrhea, acute pancreatitis, eruptive xanthomas, lipemia retinalis, and hepatosplenomegaly, or a combination thereof.

Embodiment 67. The method of embodiment 64, wherein the at least one symptom of FPL comprises chylomicronemia, abdominal pain, colicky pain, physical fatigue, difficulty thinking, diarrhea, acute pancreatitis, eruptive xanthomas, lipemia retinalis, and hepatosplenomegaly, or a combination thereof.

Embodiment 68. The method of any one of embodiments 60-67, wherein the therapeutically effective amount is 50 mg.

Embodiment 69. The method of any one of embodiments 60-67, wherein the therapeutically effective amount is 60 mg.

Embodiment 70. The method of any one of embodiments 60-67, wherein the therapeutically effective amount is 70 mg.

Embodiment 71. The method of any one of embodiments 60-67, wherein the therapeutically effective amount is 80 mg.

Embodiment 72. The method of any one of embodiments 60-67, wherein the therapeutically effective amount is any of 40 mg, 45 mg, 50 mg, 55 mg, 60 mg, 65 mg, 70 mg, 75 mg, 80 mg, 85 mg, 90 mg, 95 mg, and 100 mg.

Embodiment 73. The method of any one of embodiments 60-67, wherein the therapeutically effective amount is any of about 40 mg, about 45 mg, about 50 mg, about 55 mg, about 60 mg, about 65 mg, about 70 mg, about 75 mg, about 80 mg, about 85 mg, about 90 mg, about 95 mg, and about 100 mg.

Embodiment 74. The method of any one of embodiments 60-67, wherein the therapeutically effective amount is any of 40.0 mg, 40.1 mg, 40.2 mg, 40.3 mg, 40.4 mg, 40.5 mg, 40.6 mg, 40.7 mg, 40.8 mg, 40.9 mg, 41.0 mg, 41.1 mg, 41.2 mg, 41.3 mg, 41.4 mg, 41.5 mg, 41.6 mg, 41.7 mg, 41.8 mg, 41.9 mg, 42.0 mg, 42.1 mg, 42.2 mg, 42.3 mg, 42.4 mg, 42.5 mg, 42.6 mg, 42.7 mg, 42.8 mg, 42.9 mg, 43.0 mg, 43.1 mg, 43.2 mg, 43.3 mg, 43.4 mg, 43.5 mg, 43.6 mg, 43.7 mg, 43.8 mg, 43.9 mg, 44.0 mg, 44.1 mg, 44.2 mg, 44.3 mg, 44.4 mg, 44.5 mg, 44.6 mg, 44.7 mg, 44.8 mg, 44.9 mg, 45.0 mg, 45.1 mg, 45.2 mg, 45.3 mg, 45.4 mg, 45.5 mg, 45.6 mg, 45.7 mg, 45.8 mg, 45.9 mg, 46.0 mg, 46.1 mg, 46.2 mg, 46.3 mg, 46.4 mg, 46.5 mg, 46.6 mg, 46.7 mg, 46.8 mg, 46.9 mg, 47.0 mg, 47.1 mg, 47.2 mg, 47.3 mg, 47.4 mg, 47.5 mg, 47.6 mg, 47.7 mg, 47.8 mg, 47.9 mg, 48.0 mg, 48.1 mg, 48.2 mg, 48.3 mg, 48.4 mg, 48.5 mg, 48.6 mg, 48.7 mg, 48.8 mg, 48.9 mg, 49.0 mg, 49.1 mg, 49.2 mg, 49.3 mg, 49.4 mg, 49.5 mg, 49.6 mg, 49.7 mg, 49.8 mg, 49.9 mg, 50.0 mg, 50.1 mg, 50.2 mg, 50.3 mg, 50.4 mg, 50.5 mg, 50.6 mg, 50.7 mg, 50.8 mg, 50.9 mg, 51.0 mg, 51.1 mg, 51.2 mg, 51.3 mg, 51.4 mg, 51.5 mg, 51.6 mg, 51.7 mg, 51.8 mg, 51.9 mg, 52.0 mg, 52.1 mg, 52.2 mg, 52.3 mg, 52.4 mg, 52.5 mg, 52.6 mg, 52.7 mg, 52.8 mg, 52.9 mg, 53.0 mg, 53.1 mg, 53.2 mg, 53.3 mg. 53.4 mg, 53.5 mg, 53.6 mg, 53.7 mg, 53.8 mg, 53.9 mg, 54.0 mg, 54.1 mg, 54.2 mg, 54.3 mg, 54.4 mg, 54.5 mg, 54.6 mg, 54.7 mg, 54.8 mg, 54.9 mg, 55.0 mg, 50.0 mg, 50.1 mg, 50.2 mg, 50.3 mg, 50.4 mg, 50.5 mg, 50.6 mg, 50.7 mg, 50.8 mg, 50.9 mg, 51.0 mg, 51.1 mg, 51.2 mg, 51.3 mg, 51.4 mg, 51.5 mg, 51.6 mg, 51.7 mg, 51.8 mg, 51.9 mg, 52.0 mg, 52.1 mg, 52.2 mg, 52.3 mg, 52.4 mg, 52.5 mg, 52.6 mg, 52.7 mg, 52.8 mg, 52.9 mg, 53.0 mg, 53.1 mg, 53.2 mg, 53.3 mg. 53.4 mg, 53.5 mg, 53.6 mg, 53.7 mg, 53.8 mg, 53.9 mg, 54.0 mg, 54.1 mg, 54.2 mg, 54.3 mg, 54.4 mg, 54.5 mg, 54.6 mg, 54.7 mg, 54.8 mg, 54.9 mg, 55.0 mg, 55.1 mg, 55.2 mg, 55.3 mg. 55.4 mg, 55.5 mg, 55.6 mg, 55.7 mg, 55.8 mg, 55.9 mg, 55.0 mg, 55.1 mg, 55.2 mg, 55.3 mg, 55.4 mg, 55.5 mg, 55.6 mg, 55.7 mg, 55.8 mg, 55.9 mg, 56.0 mg, 56.1 mg, 56.2 mg, 56.3 mg. 56.4 mg, 56.5 mg, 56.6 mg, 56.7 mg, 56.8 mg, 56.9 mg, 57.0 mg, 57.1 mg, 57.2 mg, 57.3 mg, 57.4 mg, 57.5 mg, 57.6 mg, 57.7 mg, 57.8 mg, 57.9 mg, 58.0 mg, 58.1 mg, 58.2 mg, 58.3 mg. 58.4 mg, 58.5 mg, 58.6 mg, 58.7 mg, 58.8 mg, 58.9 mg, 59.0 mg, 59.1 mg, 59.2 mg, 59.3 mg, 59.4 mg, 59.5 mg, 59.6 mg, 59.7 mg, 59.8 mg, 59.9 mg, 60.0 mg, 60.1 mg, 60.2 mg, 60.3 mg, 60.4 mg, 60.5 mg, 60.6 mg, 60.7 mg, 60.8 mg, 60.9 mg, 61.0 mg, 61.1 mg, 61.2 mg, 61.3 mg, 61.4 mg, 61.5 mg, 61.6 mg, 61.7 mg, 61.8 mg, 61.9 mg, 62.0 mg, 62.1 mg, 62.2 mg, 62.3 mg, 62.4 mg, 62.5 mg, 62.6 mg, 62.7 mg, 62.8 mg, 62.9 mg, 63.0 mg, 63.1 mg, 63.2 mg, 63.3 mg, 63.4 mg, 63.5 mg, 63.6 mg, 63.7 mg, 63.8 mg, 63.9 mg, 64.0 mg, 64.1 mg, 64.2 mg, 64.3 mg, 64.4 mg, 64.5 mg, 64.6 mg, 64.7 mg, 64.8 mg, 64.9 mg, 65.0 mg, 65.1 mg, 65.2 mg, 65.3 mg, 65.4 mg, 65.5 mg, 65.6 mg, 65.7 mg, 65.8 mg, 65.9 mg, 66.0 mg, 66.1 mg, 66.2 mg, 66.3 mg, 66.4 mg, 66.5 mg, 66.6 mg, 66.7 mg, 66.8 mg, 66.9 mg, 67.0 mg, 67.1 mg, 67.2 mg, 67.3 mg, 67.4 mg, 67.5 mg, 67.6 mg, 67.7 mg, 67.8 mg, 67.9 mg, 68.0 mg, 68.1 mg, 68.2 mg, 68.3 mg, 68.4 mg, 68.5 mg, 68.6 mg, 68.7 mg, 68.8 mg, 68.9 mg, 69.0 mg, 69.1 mg, 69.2 mg, 69.3 mg, 69.4 mg, 69.5 mg, 69.6 mg, 69.7 mg, 69.8 mg, 69.9 mg, 70.0 mg, 70.1 mg, 70.2 mg, 70.3 mg, 70.4 mg, 70.5 mg, 70.6 mg, 70.7 mg, 70.8 mg, 70.9 mg, 71.0 mg, 71.1 mg, 71.2 mg, 71.3 mg, 71.4 mg, 71.5 mg, 71.6 mg, 71.7 mg, 71.8 mg, 71.9 mg, 72.0 mg, 72.1 mg, 72.2 mg, 72.3 mg, 72.4 mg, 72.5 mg, 72.6 mg, 72.7 mg, 72.8 mg, 72.9 mg, 73.0 mg, 73.1 mg, 73.2 mg, 73.3 mg, 73.4 mg, 73.5 mg, 73.6 mg, 73.7 mg, 73.8 mg, 73.9 mg, 74.0 mg, 74.1 mg, 74.2 mg, 74.3 mg, 74.4 mg, 74.5 mg, 74.6 mg, 74.7 mg, 74.8 mg, 74.9 mg, 75.0 mg, 75.1 mg, 75.2 mg, 75.3 mg, 75.4 mg, 75.5 mg, 75.6 mg, 75.7 mg, 75.8 mg, 75.9 mg, 76.0 mg, 76.1 mg, 76.2 mg, 76.3 mg, 76.4 mg, 76.5 mg, 76.6 mg, 76.7 mg, 76.8 mg, 76.9 mg, 77.0 mg, 77.1 mg, 77.2 mg, 77.3 mg, 77.4 mg, 77.5 mg, 77.6 mg, 77.7 mg, 77.8 mg, 77.9 mg, 78.0 mg, 78.1 mg, 78.2 mg, 78.3 mg, 78.4 mg, 78.5 mg, 78.6 mg, 78.7 mg, 78.8 mg, 78.9 mg, 79.0 mg, 79.1 mg, 79.2 mg, 79.3 mg, 79.4 mg, 79.5 mg, 79.6 mg, 79.7 mg, 79.8 mg, 79.9 mg, 80.0 mg, 80.1 mg, 80.2 mg, 80.3 mg, 80.4 mg, 80.5 mg, 80.6 mg, 80.7 mg, 80.8 mg, 80.9 mg, 81.0 mg, 81.1 mg, 81.2 mg, 81.3 mg, 81.4 mg, 81.5 mg, 81.6 mg, 81.7 mg, 81.8 mg, 81.9 mg, 82.0 mg, 82.0 mg, 82.1 mg, 82.2 mg, 82.3 mg, 82.4 mg, 82.5 mg, 82.6 mg, 82.7 mg, 82.8 mg, 82.9 mg, 83.0 mg, 83.1 mg, 83.2 mg, 83.3 mg, 83.4 mg, 83.5 mg, 83.6 mg, 83.7 mg, 83.8 mg, 83.9 mg, 84.0 mg, 84.1 mg, 84.2 mg, 84.3 mg, 84.4 mg, 84.5 mg, 84.6 mg, 84.7 mg, 84.8 mg, 84.9 mg, 85.0 mg, 85.0 mg, 85.1 mg, 85.2 mg, 85.3 mg, 85.4 mg, 85.5 mg, 85.6 mg, 85.7 mg, 85.8 mg, 85.9 mg, 86.0 mg, 86.1 mg, 86.2 mg, 86.3 mg, 86.4 mg, 86.5 mg, 86.6 mg, 86.7 mg, 86.8 mg, 86.9 mg, 87.0 mg, 87.1 mg, 87.2 mg, 87.3 mg, 87.4 mg, 87.5 mg, 87.6 mg, 87.7 mg, 87.8 mg, 87.9 mg, 88.0 mg, 88.1 mg, 88.2 mg, 88.3 mg, 88.4 mg, 88.5 mg, 88.6 mg, 88.7 mg, 88.8 mg, 88.9 mg, 89.0 mg, 89.0 mg, 89.1 mg, 89.2 mg, 89.3 mg, 89.4 mg, 89.5 mg, 89.6 mg, 89.7 mg, 89.8 mg, 89.9 mg, 90.0 mg, 90.1 mg, 90.2 mg, 90.3 mg, 90.4 mg, 90.5 mg, 90.6 mg, 90.7 mg, 90.8 mg, 90.9 mg, 91.0 mg, 91.1 mg, 91.2 mg, 91.3 mg, 91.4 mg, 91.5 mg, 91.6 mg, 91.7 mg, 91.8 mg, 91.9 mg, 92.0 mg, 92.0 mg, 92.1 mg, 92.2 mg, 92.3 mg, 92.4 mg, 92.5 mg, 92.6 mg, 92.7 mg, 92.8 mg, 92.9 mg, 93.0 mg, 93.1 mg, 93.2 mg, 93.3 mg, 93.4 mg, 93.5 mg, 93.6 mg, 93.7 mg, 93.8 mg, 93.9 mg, 94.0 mg, 94.1 mg, 94.2 mg, 94.3 mg, 94.4 mg, 94.5 mg, 94.6 mg, 94.7 mg, 94.8 mg, 94.9 mg, 95.0 mg, 95.1 mg, 95.2 mg, 95.3 mg, 95.4 mg, 95.5 mg, 95.6 mg, 95.7 mg, 95.8 mg, 95.9 mg, 96.0 mg, 96.1 mg, 96.2 mg, 96.3 mg, 96.4 mg, 96.5 mg, 96.6 mg, 96.7 mg, 96.8 mg, 96.9 mg, 97.0 mg, 97.1 mg, 97.2 mg, 97.3 mg, 97.4 mg, 97.5 mg, 97.6 mg, 97.7 mg, 97.8 mg, 97.9 mg, 98.0 mg, 98.1 mg, 98.2 mg, 98.3 mg, 98.4 mg, 98.5 mg, 98.6 mg, 98.7 mg, 98.8 mg, 98.9 mg, 99.0 mg, 99.1 mg, 99.2 mg, 99.3 mg, 99.4 mg, 99.5 mg, 99.6 mg, 99.7 mg, 99.8 mg, 99.9 mg, and 100.0 mg.

Embodiment 75. The method of any one of embodiments 60-67, wherein the therapeutically effective amount is any of about 40.0 mg, about 40.1 mg, about 40.2 mg, about 40.3 mg, about 40.4 mg, about 40.5 mg, about 40.6 mg, about 40.7 mg, about 40.8 mg, about 40.9 mg, about 41.0 mg, about 41.1 mg, about 41.2 mg, about 41.3 mg, about 41.4 mg, about 41.5 mg, about 41.6 mg, about 41.7 mg, about 41.8 mg, about 41.9 mg, about 42.0 mg, about 42.1 mg, about 42.2 mg, about 42.3 mg, about 42.4 mg, about 42.5 mg, about 42.6 mg, about 42.7 mg, about 42.8 mg, about 42.9 mg, about 43.0 mg, about 43.1 mg, about 43.2 mg, about 43.3 mg, about 43.4 mg, about 43.5 mg, about 43.6 mg, about 43.7 mg, about 43.8 mg, about 43.9 mg, about 44.0 mg, about 44.1 mg, about 44.2 mg, about 44.3 mg, about 44.4 mg, about 44.5 mg, about 44.6 mg, about 44.7 mg, about 44.8 mg, about 44.9 mg, about 45.0 mg, about 45.1 mg, about 45.2 mg, about 45.3 mg, about 45.4 mg, about 45.5 mg, about 45.6 mg, about 45.7 mg, about 45.8 mg, about 45.9 mg, about 46.0 mg, about 46.1 mg, about 46.2 mg, about 46.3 mg, about 46.4 mg, about 46.5 mg, about 46.6 mg, about 46.7 mg, about 46.8 mg, about 46.9 mg, about 47.0 mg, about 47.1 mg, about 47.2 mg, about 47.3 mg, about 47.4 mg, about 47.5 mg, about 47.6 mg, about 47.7 mg, about 47.8 mg, about 47.9 mg, about 48.0 mg, about 48.1 mg, about 48.2 mg, about 48.3 mg, about 48.4 mg, about 48.5 mg, about 48.6 mg, about 48.7 mg, about 48.8 mg, about 48.9 mg, about 49.0 mg, about 49.1 mg, about 49.2 mg, about 49.3 mg, about 49.4 mg, about 49.5 mg, about 49.6 mg, about 49.7 mg, about 49.8 mg, about 49.9 mg, about 50.0 mg, about 50.1 mg, about 50.2 mg, about 50.3 mg, about 50.4 mg, about 50.5 mg, about 50.6 mg, about 50.7 mg, about 50.8 mg, about 50.9 mg, about 51.0 mg, about 51.1 mg, about 51.2 mg, about 51.3 mg, about 51.4 mg, about 51.5 mg, about 51.6 mg, about 51.7 mg, about 51.8 mg, about 51.9 mg, about 52.0 mg, about 52.1 mg, about 52.2 mg, about 52.3 mg, about 52.4 mg, about 52.5 mg, about 52.6 mg, about 52.7 mg, about 52.8 mg, about 52.9 mg, about 53.0 mg, about 53.1 mg, about 53.2 mg, about 53.3 mg. about 53.4 mg, about 53.5 mg, about 53.6 mg, about 53.7 mg, about 53.8 mg, about 53.9 mg, about 54.0 mg, about 54.1 mg, about 54.2 mg, about 54.3 mg, about 54.4 mg, about 54.5 mg, about 54.6 mg, about 54.7 mg, about 54.8 mg, about 54.9 mg, about 55.0 mg, about 55.1 mg, about 55.2 mg, about 55.3 mg. about 55.4 mg, about 55.5 mg, about 55.6 mg, about 55.7 mg, about 55.8 mg, about 55.9 mg, about 55.0 mg, about 55.1 mg, about 55.2 mg, about 55.3 mg, about 55.4 mg, about 55.5 mg, about 55.6 mg, about 55.7 mg, about 55.8 mg, about 55.9 mg, about 56.0 mg, about 56.1 mg, about 56.2 mg, about 56.3 mg. about 56.4 mg, about 56.5 mg, about 56.6 mg, about 56.7 mg, about 56.8 mg, about 56.9 mg, about 57.0 mg, about 57.1 mg, about 57.2 mg, about 57.3 mg, about 57.4 mg, about 57.5 mg, about 57.6 mg, about 57.7 mg, about 57.8 mg, about 57.9 mg, about 58.0 mg, about 58.1 mg, about 58.2 mg, about 58.3 mg. about 58.4 mg, about 58.5 mg, about 58.6 mg, about 58.7 mg, about 58.8 mg, about 58.9 mg, about 59.0 mg, about 59.1 mg, about 59.2 mg, about 59.3 mg, about 59.4 mg, about 59.5 mg, about 59.6 mg, about 59.7 mg, about 59.8 mg, about 59.9 mg, about 60.0 mg, about 60.1 mg, about 60.2 mg, about 60.3 mg, about 60.4 mg, about 60.5 mg, about 60.6 mg, about 60.7 mg, about 60.8 mg, about 60.9 mg, about 61.0 mg, about 61.1 mg, about 61.2 mg, about 61.3 mg, about 61.4 mg, about 61.5 mg, about 61.6 mg, about 61.7 mg, about 61.8 mg, about 61.9 mg, about 62.0 mg, about 62.1 mg, about 62.2 mg, about 62.3 mg, about 62.4 mg, about 62.5 mg, about 62.6 mg, about 62.7 mg, about 62.8 mg, about 62.9 mg, about 63.0 mg, about 63.1 mg, about 63.2 mg, about 63.3 mg, about 63.4 mg, about 63.5 mg, about 63.6 mg, about 63.7 mg, about 63.8 mg, about 63.9 mg, about 64.0 mg, about 64.1 mg, about 64.2 mg, about 64.3 mg, about 64.4 mg, about 64.5 mg, about 64.6 mg, about 64.7 mg, about 64.8 mg, about 64.9 mg, about 65.0 mg, about 65.1 mg, about 65.2 mg, about 65.3 mg, about 65.4 mg, about 65.5 mg, about 65.6 mg, about 65.7 mg, about 65.8 mg, about 65.9 mg, about 66.0 mg, about 66.1 mg, about 66.2 mg, about 66.3 mg, about 66.4 mg, about 66.5 mg, about 66.6 mg, about 66.7 mg, about 66.8 mg, about 66.9 mg, about 67.0 mg, about 67.1 mg, about 67.2 mg, about 67.3 mg, about 67.4 mg, about 67.5 mg, about 67.6 mg, about 67.7 mg, about 67.8 mg, about 67.9 mg, about 68.0 mg, about 68.1 mg, about 68.2 mg, about 68.3 mg, about 68.4 mg, about 68.5 mg, about 68.6 mg, about 68.7 mg, about 68.8 mg, about 68.9 mg, about 69.0 mg, about 69.1 mg, about 69.2 mg, about 69.3 mg, about 69.4 mg, about 69.5 mg, about 69.6 mg, about 69.7 mg, about 69.8 mg, about 69.9 mg, about 70.0 mg, about 70.1 mg, about 70.2 mg, about 70.3 mg, about 70.4 mg, about 70.5 mg, about 70.6 mg, about 70.7 mg, about 70.8 mg, about 70.9 mg, about 71.0 mg, about 71.1 mg, about 71.2 mg, about 71.3 mg, about 71.4 mg, about 71.5 mg, about 71.6 mg, about 71.7 mg, about 71.8 mg, about 71.9 mg, about 72.0 mg, about 72.1 mg, about 72.2 mg, about 72.3 mg, about 72.4 mg, about 72.5 mg, about 72.6 mg, about 72.7 mg, about 72.8 mg, about 72.9 mg, about 73.0 mg, about 73.1 mg, about 73.2 mg, about 73.3 mg, about 73.4 mg, about 73.5 mg, about 73.6 mg, about 73.7 mg, about 73.8 mg, about 73.9 mg, about 74.0 mg, about 74.1 mg, about 74.2 mg, about 74.3 mg, about 74.4 mg, about 74.5 mg, about 74.6 mg, about 74.7 mg, about 74.8 mg, about 74.9 mg, about 75.0 mg, about 75.1 mg, about 75.2 mg, about 75.3 mg, about 75.4 mg, about 75.5 mg, about 75.6 mg, about 75.7 mg, about 75.8 mg, about 75.9 mg, about 76.0 mg, about 76.1 mg, about 76.2 mg, about 76.3 mg, about 76.4 mg, about 76.5 mg, about 76.6 mg, about 76.7 mg, about 76.8 mg, about 76.9 mg, about 77.0 mg, about 77.1 mg, about 77.2 mg, about 77.3 mg, about 77.4 mg, about 77.5 mg, about 77.6 mg, about 77.7 mg, about 77.8 mg, about 77.9 mg, about 78.0 mg, about 78.1 mg, about 78.2 mg, about 78.3 mg, about 78.4 mg, about 78.5 mg, about 78.6 mg, about 78.7 mg, about 78.8 mg, about 78.9 mg, about 79.0 mg, about 79.1 mg, about 79.2 mg, about 79.3 mg, about 79.4 mg, about 79.5 mg, about 79.6 mg, about 79.7 mg, about 79.8 mg, about 79.9 mg, about 80.0 mg, about 80.1 mg, about 80.2 mg, about 80.3 mg, about 80.4 mg, about 80.5 mg, about 80.6 mg, about 80.7 mg, about 80.8 mg, about 80.9 mg, about 81.0 mg, about 81.1 mg, about 81.2 mg, about 81.3 mg, about 81.4 mg, about 81.5 mg, about 81.6 mg, about 81.7 mg, about 81.8 mg, about 81.9 mg, about 82.0 mg, about 82.0 mg, about 82.1 mg, about 82.2 mg, about 82.3 mg, about 82.4 mg, about 82.5 mg, about 82.6 mg, about 82.7 mg, about 82.8 mg, about 82.9 mg, about 83.0 mg, about 83.1 mg, about 83.2 mg, about 83.3 mg, about 83.4 mg, about 83.5 mg, about 83.6 mg, about 83.7 mg, about 83.8 mg, about 83.9 mg, about 84.0 mg, about 84.1 mg, about 84.2 mg, about 84.3 mg, about 84.4 mg, about 84.5 mg, about 84.6 mg, about 84.7 mg, about 84.8 mg, about 84.9 mg, about 85.0 mg, about 85.1 mg, about 85.2 mg, about 85.3 mg, about 85.4 mg, about 85.5 mg, about 85.6 mg, about 85.7 mg, about 85.8 mg, about 85.9 mg, about 86.0 mg, about 86.1 mg, about 86.2 mg, about 86.3 mg, about 86.4 mg, about 86.5 mg, about 86.6 mg, about 86.7 mg, about 86.8 mg, about 86.9 mg, about 87.0 mg, about 87.1 mg, about 87.2 mg, about 87.3 mg, about 87.4 mg, about 87.5 mg, about 87.6 mg, about 87.7 mg, about 87.8 mg, about 87.9 mg, about 88.0 mg, about 88.1 mg, about 88.2 mg, about 88.3 mg, about 88.4 mg, about 88.5 mg, about 88.6 mg, about 88.7 mg, about 88.8 mg, about 88.9 mg, about 89.0 mg, about 89.1 mg, about 89.2 mg, about 89.3 mg, about 89.4 mg, about 89.5 mg, about 89.6 mg, about 89.7 mg, about 89.8 mg, about 89.9 mg, about 90.0 mg, about 90.1 mg, about 90.2 mg, about 90.3 mg, about 90.4 mg, about 90.5 mg, about 90.6 mg, about 90.7 mg, about 90.8 mg, about 90.9 mg, about 91.0 mg, about 91.1 mg, about 91.2 mg, about 91.3 mg, about 91.4 mg, about 91.5 mg, about 91.6 mg, about 91.7 mg, about 91.8 mg, about 91.9 mg, about 92.0 mg, about 92.1 mg, about 92.2 mg, about 92.3 mg, about 92.4 mg, about 92.5 mg, about 92.6 mg, about 92.7 mg, about 92.8 mg, about 92.9 mg, about 93.0 mg, about 93.1 mg, about 93.2 mg, about 93.3 mg, about 93.4 mg, about 93.5 mg, about 93.6 mg, about 93.7 mg, about 93.8 mg, about 93.9 mg, about 94.0 mg, about 94.1 mg, about 94.2 mg, about 94.3 mg, about 94.4 mg, about 94.5 mg, about 94.6 mg, about 94.7 mg, about 94.8 mg, about 94.9 mg, about 95.0 mg, about 95.1 mg, about 95.2 mg, about 95.3 mg, about 95.4 mg, about 95.5 mg, about 95.6 mg, about 95.7 mg, about 95.8 mg, about 95.9 mg, about 96.0 mg, about 96.1 mg, about 96.2 mg, about 96.3 mg, about 96.4 mg, about 96.5 mg, about 96.6 mg, about 96.7 mg, about 96.8 mg, about 96.9 mg, about 97.0 mg, about 97.1 mg, about 97.2 mg, about 97.3 mg, about 97.4 mg, about 97.5 mg, about 97.6 mg, about 97.7 mg, about 97.8 mg, about 97.9 mg, about 98.0 mg, about 98.1 mg, about 98.2 mg, about 98.3 mg, about 98.4 mg, about 98.5 mg, about 98.6 mg, about 98.7 mg, about 98.8 mg, about 98.9 mg, about 99.0 mg, about 99.1 mg, about 99.2 mg, about 99.3 mg, about 99.4 mg, about 99.5 mg, about 99.6 mg, about 99.7 mg, about 99.8 mg, about 99.9 mg, and about 100.0 mg.

Embodiment 76. The method of any one of embodiments 60-67, wherein the therapeutically effective amount is within the range of any of 40 mg to 100 mg, 40 mg to 80 mg, 40 mg to 70 mg, 40 mg to 60 mg, 40 mg to 50 mg, 50 mg to 100 mg, 50 mg to 80 mg, 50 mg to 70 mg, 50 mg to 60 mg, 60 mg to 100 mg, 60 mg to 80 mg, 60 mg to 70 mg, 70 mg to 100 mg, 70 mg to 80 mg, 80 mg to 100 mg, 80 mg to 90 mg, and 90 mg to 100 mg.

Embodiment 77. The method of any one of embodiments 60-67, wherein the therapeutically effective amount is any of less than 100 mg, less than 95 mg, less than 90 mg, less than 85 mg, less than 80 mg, less than 75 mg, less than 70 mg, less than 65 mg, less than 60 mg, less than 55 mg, less than 50 mg, less than 45 mg, and less than 40 mg.

Embodiment 78. The method of any one of embodiments 60-67, wherein the therapeutically effective amount is any of less than about less than about 100 mg, less than about 95 mg, less than about 90 mg, less than about 85 mg, less than about 80 mg, less than about 75 mg, less than about 70 mg, less than about 65 mg, less than about 60 mg, less than about 55 mg, less than about 50 mg, less than about 45 mg, and less than about 40 mg.

Embodiment 79. The method of any one of embodiments 60-67, wherein the therapeutically effective amount is any of at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, and at least about 100 mg.

Embodiment 80. The method of any one of embodiments 60-67, wherein the therapeutically effective amount is any of at least about 40 mg, at least about 45 mg, at least about 50 mg, at least about 55 mg, at least about 60 mg, at least about 65 mg, at least about 70 mg, at least about 75 mg, at least about 80 mg, at least about 85 mg, at least about 90 mg, at least about 95 mg, and at least about 100 mg.

Embodiment 81. The method of any one of embodiments 60-80, comprising administering the modified oligonucleotide once every 4 weeks.

Embodiment 82. The method of any one of embodiments 60-80, comprising administering the modified oligonucleotide once every 8 weeks.

Embodiment 83. The method of any one of embodiments 60-80, comprising administering the modified oligonucleotide once every 12 weeks.

Embodiment 84. The method of any one of embodiments 60-80, comprising administering the modified oligonucleotide once every 16 weeks.

Embodiment 85. The method of any one of embodiments 60-80, comprising administering the modified oligonucleotide once every 20 weeks.

Embodiment 86. The method of any one of embodiments 60-80, comprising administering the modified oligonucleotide about once every 4 weeks.

Embodiment 87. The method of any one of embodiments 60-80, comprising administering the modified oligonucleotide about once every 8 weeks.

Embodiment 88. The method of any one of embodiments 60-80, comprising administering the modified oligonucleotide about once every 12 weeks.

Embodiment 89. The method of any one of embodiments 60-80, comprising administering the modified oligonucleotide about once every 16 weeks.

Embodiment 90. The method of any one of embodiments 60-80, comprising administering the modified oligonucleotide about once every 20 weeks.

Embodiment 91. The method of any one of embodiments 60-80, comprising administering the modified oligonucleotide any of once every 1 week, once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks, once every 17 weeks, once every 18 weeks, once every 19 weeks, and once every 20 weeks.

Embodiment 92. The method of any one of embodiments 60-80, comprising administering the modified oligonucleotide any of once about every 1 week, once about every 2 weeks, once about every 3 weeks, once about every 4 weeks, once about every 5 weeks, once about every 6 weeks, once about every 7 weeks, once about every 8 weeks, once about every 9 weeks, once about every 10 weeks, once about every 11 weeks, once about every 12 weeks, once about every 13 weeks, once about every 14 weeks, once about every 15 weeks, once about every 16 weeks, once about every 17 weeks, once about every 18 weeks, once about every 19 weeks, and once about every 20 weeks.

Embodiment 93. The method of any one of embodiments 60-92, wherein the subject has FCS.

Embodiment 94. The method of any one of embodiments 60-92, wherein the subject has FPL.

Embodiment 95. The method of any one of embodiments 60-94, wherein the subject has STHG.

I. APOCIII

In certain embodiments, described herein are methods of reducing APOCIII RNA and/or APOCIII protein in a cell or a biological fluid of a subject. APOCIII RNA is encoded by the human APOCIII gene. APOCIII protein is the protein expression product of APOCIII RNA. A representative nucleobase sequence for a human APOCIII gene is provided at GENBANK Accession No. NT_035088.1 truncated from nucleobases 6238608 to 6242565, incorporated herein as SEQ ID NO: 1. A representative nucleobase sequence for a human APOCIII RNA is provided at GENBANK Accession No. NM_000040.2, incorporated herein as SEQ ID NO: 2.

II. ISIS 678354

In certain embodiments, described herein are methods of administering modified oligonucleotide, ISIS 678354, to a subject in need thereof. In certain embodiments, ISIS 678354 is characterized as a 5-10-5 MOE gapmer covalently bound at the 5′-end to triantenary N-acetyl galactosamine (GalNAc₃), a high affinity ligand for the hepatocyte-specific asialoglycoprotein receptor (ASGPR) (Prakash T P, Graham M J, Yu J, et al Nucleic Acids Res 2014; 42: 8796-8807). ISIS 678354 has a sequence of (from 5′ to 3′) AGCTTCTTGTCCAGCTTTAT (incorporated herein as SEQ ID NO: 3), wherein each of nucleosides 1-5 and 16-20 (from 5′ to 3′) are 2′-MOE nucleosides and each of nucleosides 6-15 are 2′-β-D deoxyribonucleosides, wherein the internucleoside linkages are phosphorothioate internucleoside linkages, and wherein each cytosine is a 5-methyl cytosine. ISIS 678354 has a 5′-trishexylamino-(THA)-C₆GalNAc₃ endcap, represented by the structure below, wherein the phosphate group is attached to the 5′-oxygen atom of the 5′-nucleoside:

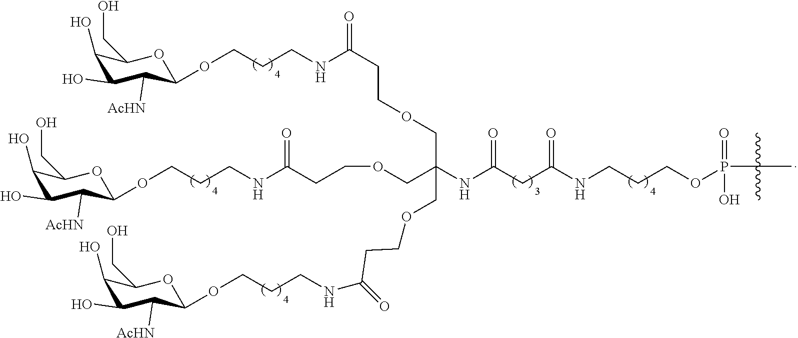

In certain embodiments, ISIS 678354 is represented by the following chemical notation (5′ to 3′): THA-C6-GalNAc₃ AₑₛGₑₛ^{m}CₑₛTₑₛTₑₛ^{m}C_{ds}T_{ds}T_{ds}G_{ds}T_{ds}^{m}C_{ds}^{m}C_{ds}A_{ds}G_{ds}^{m}C_{ds}TₑₛTₑₛTₑₛAₑₛTₑ (SEQ ID NO: 3); wherein,
A=an adenine nucleobase,
mC=a 5-methyl cytosine nucleobase,
G=a guanine nucleobase,
T=a thymine nucleobase,
e=a 2′-MOE sugar moiety,
d=a 2′-β-D-deoxyribosyl sugar moiety, and
s=a phosphorothioate internucleoside linkage.

In certain embodiments, ISIS 678354 is represented by the following chemical structure:

(SEQ ID NO: 3) or a salt thereof.

In certain embodiments, the sodium salt of ISIS 678354 is represented by the following chemical structure:

Previous Clinical Trials

### Phase 1

ISIS 678354-CS1 was a Phase 1, double-blind, placebo-controlled, dose-escalation study designed to assess the safety, tolerability and pharmacokinetics of single and multiple doses of ISIS 678354 administered by subcutaneous (SC) injection to healthy subjects (age 18 to 65). In the single-ascending dose (SAD) part of the study, 5 dose levels (10, 30, 60, 90, and 120 mg) were evaluated sequentially. In the multiple-ascending dose (MAD) part, 2 dose levels (15 and 30 mg) were evaluated at weekly dosing for 6 weeks and 1 dose level (60 mg) was evaluated at every-4-week dosing for 3 months. The SAD and weekly MAD dose levels were studied in a cohort of 8 subjects, where 6 were randomized to active treatment with ISIS 678354 and 2 were randomized to placebo. In the every-4-week MAD cohort 10 subjects were studied (6 active and 4 placebo). In the 90 and 120 mg SAD and all of the MAD cohorts, the pharmacodynamic effects of ISIS 678354 vs. placebo were examined in healthy subjects with elevated triglycerides (TG >200 mg/dL and ≤500 mg/dL).

Results from the SAD portion of the Phase 1 study showed significant dose-dependent sustained reductions in fasting total apoC-III and TG levels. Significant dose-dependent reductions of up to ˜−30% in apoB and increases of up to ˜100% in HDL-C were also observed. There were no notable increases in LDL-C in any dose group. Effects were sustained for at least 4 weeks after the last dose, consistent with the drug's long terminal elimination half-life. Overall, ISIS 678354 was well-tolerated and there were no safety concerns.

### Phase 2

The Phase 2 study was a multicenter, randomized, double-blind, placebo-controlled, dose-ranging study. A total of 114 patients with established cardiovascular disease (CVD), or being at risk for CVD, with fasting TG levels of ≥200 mg/dL and ≤500 mg/dL, and on standard-of-care preventative therapy for their known CVD risk factors were randomly assigned to 1 of the 4 parallel dosing cohorts, with each cohort having a 4:1 ratio to receive ISIS 678354 or matching volume of placebo, respectively, by SC injection for up to 12 months. The study evaluated 3 different doses of ISIS 678354: 10, 15, and 50 mg, and 3 different dosing regimens: weekly, every 2 weeks, and every 4 weeks (Q4W). The range of dosing covered the equivalent monthly drug exposure of 10 mg to 50 mg. Treatment duration was 6 to 12 months. The treatment portion of the study was complete when the last patient reached 6 months of exposure. All patients then entered a 13-week Post-Treatment Follow-up Period.

The primary efficacy analysis for the primary endpoint was the pairwise comparison of percent change from Baseline to the primary analysis time point in fasting TG between ISIS 678354-treated groups and pooled-placebo group. The primary efficacy analysis time point was at Week 25 for patients who received every 4-week dosing and at Week 27 for patients who received every 2-week or weekly dosing.

Significant reductions in fasting TG levels from Baseline to the primary analysis time point were observed in all ISIS 678354-treated groups compared to placebo. The highest dose group (50 mg Q4W) achieved mean 62% reduction from Baseline in fasting TG levels compared to placebo, associated with mean 74% reduction from Baseline in apoC-III levels. The proportion of patients achieving normal fasting TG levels <150 mg/dL showed a significant, dose-dependent increase in all ISIS 678354 treated groups with over 90% (20/22) patients treated with the highest dose of 50 mg Q4W vs. only 4.2% (1/24) patients treated with placebo achieving this threshold. ISIS 678354 was well-tolerated and there were no safety concerns.

III. Certain Pharmaceutical Compositions

In certain embodiments, described herein are methods of administering to a subject a pharmaceutical composition comprising the modified oligonucleotide, ISIS 678354. In certain embodiments, the pharmaceutical composition comprises a pharmaceutically acceptable diluent or carrier. In certain embodiments, the pharmaceutical composition comprises or consists essentially of a sterile saline solution and the modified oligonucleotide ISIS 678354. In certain embodiments, the sterile saline is pharmaceutical grade saline. In certain embodiments, the pharmaceutical composition comprises or consists essentially of sterile water and the modified oligonucleotide ISIS 678354. In certain embodiments, the sterile water is pharmaceutical grade water. In certain embodiments, the pharmaceutically acceptable diluent or carrier is water or saline. The water may be water for injection (WFI). The saline may be phosphate-buffered saline.

In certain embodiments, pharmaceutical compositions comprise one or more excipients and the modified oligonucleotide ISIS 678354. In certain embodiments, excipients are selected from water, salt solutions, alcohol, polyethylene glycols, gelatin, lactose, amylase, magnesium stearate, talc, silicic acid, viscous paraffin, hydroxymethylcellulose, and polyvinylpyrrolidone.

In certain embodiments, pharmaceutical compositions comprising the modified oligonucleotide ISIS 678354 encompass any pharmaceutically acceptable salt of the modified oligonucleotide ISIS 678354, esters of the modified oligonucleotide ISIS 678354, or salts of such esters. In certain embodiments, pharmaceutical compositions comprising the modified oligonucleotide ISIS 678354 are capable of providing (directly or indirectly) the biologically active metabolite or residue thereof upon administration to a human subject. Accordingly, for example, the disclosure is also drawn to pharmaceutically acceptable salts of the modified oligonucleotide ISIS 678354, prodrugs of the modified oligonucleotide ISIS 678354, pharmaceutically acceptable salts of such prodrugs, and other bioequivalents. Suitable pharmaceutically acceptable salts include, but are not limited to, sodium and potassium salts.

In certain embodiments, pharmaceutical compositions comprise one or more lipid moieties and the modified oligonucleotide ISIS 678354. In certain embodiments, lipid moieties are used to increase distribution of ISIS 678354 to a particular cell or tissue. In certain such methods, the modified oligonucleotide ISIS 678354 is introduced into preformed liposomes or lipoplexes made of mixtures of cationic lipids and neutral lipids. In certain methods, DNA complexes with mono- or poly-cationic lipids are formed without the presence of a neutral lipid.

In certain embodiments, pharmaceutical compositions disclosed herein comprise a delivery system. Examples of delivery systems include, but are not limited to, liposomes and emulsions. Certain delivery systems are useful for preparing pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver modified oligonucleotides described herein to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, a pharmaceutical composition is prepared for administration by injection (e.g., intravenous, subcutaneous, intramuscular, intrathecal (IT), intracerebroventricular (ICV)). In certain of such embodiments, a pharmaceutical composition comprises a carrier and is formulated in aqueous solution, such as aCSF, water, or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

Under certain conditions, the modified oligonucleotide ISIS 678354 acts as an acid. Although ISIS 678354 may be drawn or described in protonated (free acid) form, or ionized and in association with a cation (salt) form, aqueous solutions of ISIS 678354 exist in equilibrium among such forms. For example, a phosphate linkage of ISIS 678354 in aqueous solution exists in equilibrium among free acid, anion, and salt forms. Unless otherwise indicated, the term, “ISIS 678354,” is intended to include all such forms. Moreover, ISIS 678354 has several such linkages, each of which is in equilibrium. Thus, ISIS 678354 exists in solution in an ensemble of forms at multiple positions all at equilibrium. The term “ISIS 678354” is intended to include all such forms. Drawn structures necessarily depict a single form. Nevertheless, unless otherwise indicated, such drawings are likewise intended to include corresponding forms. Herein, a structure depicting the free acid of ISIS 678354 followed by the term “or a salt thereof” expressly includes all such forms that may be fully or partially protonated/de-protonated/in association with a cation. In certain instances, one or more specific cation is identified.

In certain embodiments, ISIS 678354 is in aqueous solution with sodium. In certain embodiments, ISIS 678354 is in aqueous solution with potassium. In certain embodiments, ISIS 678354 is in PBS. In certain embodiments, ISIS 678354 is in water. In certain such embodiments, the pH of the solution is adjusted with NaOH and/or HCl to achieve a desired pH.

Herein, certain specific doses are described. For clarity, a dose of ISIS 678354 in milligrams indicates the mass of the free acid form of ISIS 678354. As described above, in aqueous solution, the free acid is in equilibrium with anionic and salt forms. However, for the purpose of calculating dose, it is assumed that ISIS 678354 exists as a solvent-free, sodium-acetate free, anhydrous, free acid. For example, where ISIS 678354 is in solution comprising sodium (e.g., saline), ISIS 678354 may be partially or fully de-protonated and in association with Na+ ions. However, the mass of the protons is nevertheless counted toward the weight of the dose, and the mass of the Na+ ions are not counted toward the weight of the dose. Thus, for example, a dose of 80 mg of ISIS 678354 equals the number of fully protonated molecules that weighs 80 mg.

In certain embodiments, administration is subcutaneous.

IV. Certain Dosage Amounts

In certain embodiments, described herein are methods of administering to a subject a therapeutically effective amount of the modified oligonucleotide ISIS 678354. The amount may be therapeutically effective in treating or ameliorating a human subject having a disease or condition described herein, for example, Familial Chylomicronemia Syndrome (FCS), severe hypertriglyceridemia (SHTG), or Familial Partial Lipodystrophy (FPL). In certain embodiments, the amount is therapeutically effective in treating Familial Chylomicronemia Syndrome (FCS). In certain embodiments, the therapeutically effective amount is 50 mg. In certain embodiments, the therapeutically effective amount is 60 mg. In certain embodiments, the therapeutically effective amount is 70 mg. In certain embodiments, the therapeutically effective amount is 80 mg.

In certain embodiments, the therapeutically effective amount is any of 40 mg, 45 mg, 50 mg, 55 mg, 60 mg, 65 mg, 70 mg, 75 mg, 80 mg, 85 mg, 90 mg, 95 mg, and 100 mg.

In certain embodiments, the therapeutically effective amount is any of about 40 mg, about 45 mg, about 50 mg, about 55 mg, about 60 mg, about 65 mg, about 70 mg, about 75 mg, about 80 mg, about 85 mg, about 90 mg, about 95 mg, and about 100 mg.

In certain embodiments, the therapeutically effective amount is any of 40.0 mg, 40.1 mg, 40.2 mg, 40.3 mg, 40.4 mg, 40.5 mg, 40.6 mg, 40.7 mg, 40.8 mg, 40.9 mg, 41.0 mg, 41.1 mg, 41.2 mg, 41.3 mg, 41.4 mg, 41.5 mg, 41.6 mg, 41.7 mg, 41.8 mg, 41.9 mg, 42.0 mg, 42.1 mg, 42.2 mg, 42.3 mg, 42.4 mg, 42.5 mg, 42.6 mg, 42.7 mg, 42.8 mg, 42.9 mg, 43.0 mg, 43.1 mg, 43.2 mg, 43.3 mg, 43.4 mg, 43.5 mg, 43.6 mg, 43.7 mg, 43.8 mg, 43.9 mg, 44.0 mg, 44.1 mg, 44.2 mg, 44.3 mg, 44.4 mg, 44.5 mg, 44.6 mg, 44.7 mg, 44.8 mg, 44.9 mg, 45.0 mg, 45.1 mg, 45.2 mg, 45.3 mg, 45.4 mg, 45.5 mg, 45.6 mg, 45.7 mg, 45.8 mg, 45.9 mg, 46.0 mg, 46.1 mg, 46.2 mg, 46.3 mg, 46.4 mg, 46.5 mg, 46.6 mg, 46.7 mg, 46.8 mg, 46.9 mg, 47.0 mg, 47.1 mg, 47.2 mg, 47.3 mg, 47.4 mg, 47.5 mg, 47.6 mg, 47.7 mg, 47.8 mg, 47.9 mg, 48.0 mg, 48.1 mg, 48.2 mg, 48.3 mg, 48.4 mg, 48.5 mg, 48.6 mg, 48.7 mg, 48.8 mg, 48.9 mg, 49.0 mg, 49.1 mg, 49.2 mg, 49.3 mg, 49.4 mg, 49.5 mg, 49.6 mg, 49.7 mg, 49.8 mg, 49.9 mg, 50.0 mg, 50.1 mg, 50.2 mg, 50.3 mg, 50.4 mg, 50.5 mg, 50.6 mg, 50.7 mg, 50.8 mg, 50.9 mg, 51.0 mg, 51.1 mg, 51.2 mg, 51.3 mg, 51.4 mg, 51.5 mg, 51.6 mg, 51.7 mg, 51.8 mg, 51.9 mg, 52.0 mg, 52.1 mg, 52.2 mg, 52.3 mg, 52.4 mg, 52.5 mg, 52.6 mg, 52.7 mg, 52.8 mg, 52.9 mg, 53.0 mg, 53.1 mg, 53.2 mg, 53.3 mg. 53.4 mg, 53.5 mg, 53.6 mg, 53.7 mg, 53.8 mg, 53.9 mg, 54.0 mg, 54.1 mg, 54.2 mg, 54.3 mg, 54.4 mg, 54.5 mg, 54.6 mg, 54.7 mg, 54.8 mg, 54.9 mg, 55.0 mg, 50.0 mg, 50.1 mg, 50.2 mg, 50.3 mg, 50.4 mg, 50.5 mg, 50.6 mg, 50.7 mg, 50.8 mg, 50.9 mg, 51.0 mg, 51.1 mg, 51.2 mg, 51.3 mg, 51.4 mg, 51.5 mg, 51.6 mg, 51.7 mg, 51.8 mg, 51.9 mg, 52.0 mg, 52.1 mg, 52.2 mg, 52.3 mg, 52.4 mg, 52.5 mg, 52.6 mg, 52.7 mg, 52.8 mg, 52.9 mg, 53.0 mg, 53.1 mg, 53.2 mg, 53.3 mg. 53.4 mg, 53.5 mg, 53.6 mg, 53.7 mg, 53.8 mg, 53.9 mg, 54.0 mg, 54.1 mg, 54.2 mg, 54.3 mg, 54.4 mg, 54.5 mg, 54.6 mg, 54.7 mg, 54.8 mg, 54.9 mg, 55.0 mg, 55.1 mg, 55.2 mg, 55.3 mg. 55.4 mg, 55.5 mg, 55.6 mg, 55.7 mg, 55.8 mg, 55.9 mg, 55.0 mg, 55.1 mg, 55.2 mg, 55.3 mg, 55.4 mg, 55.5 mg, 55.6 mg, 55.7 mg, 55.8 mg, 55.9 mg, 56.0 mg, 56.1 mg, 56.2 mg, 56.3 mg. 56.4 mg, 56.5 mg, 56.6 mg, 56.7 mg, 56.8 mg, 56.9 mg, 57.0 mg, 57.1 mg, 57.2 mg, 57.3 mg, 57.4 mg, 57.5 mg, 57.6 mg, 57.7 mg, 57.8 mg, 57.9 mg, 58.0 mg, 58.1 mg, 58.2 mg, 58.3 mg. 58.4 mg, 58.5 mg, 58.6 mg, 58.7 mg, 58.8 mg, 58.9 mg, 59.0 mg, 59.1 mg, 59.2 mg, 59.3 mg, 59.4 mg, 59.5 mg, 59.6 mg, 59.7 mg, 59.8 mg, 59.9 mg, 60.0 mg, 60.1 mg, 60.2 mg, 60.3 mg, 60.4 mg, 60.5 mg, 60.6 mg, 60.7 mg, 60.8 mg, 60.9 mg, 61.0 mg, 61.1 mg, 61.2 mg, 61.3 mg, 61.4 mg, 61.5 mg, 61.6 mg, 61.7 mg, 61.8 mg, 61.9 mg, 62.0 mg, 62.1 mg, 62.2 mg, 62.3 mg, 62.4 mg, 62.5 mg, 62.6 mg, 62.7 mg, 62.8 mg, 62.9 mg, 63.0 mg, 63.1 mg, 63.2 mg, 63.3 mg, 63.4 mg, 63.5 mg, 63.6 mg, 63.7 mg, 63.8 mg, 63.9 mg, 64.0 mg, 64.1 mg, 64.2 mg, 64.3 mg, 64.4 mg, 64.5 mg, 64.6 mg, 64.7 mg, 64.8 mg, 64.9 mg, 65.0 mg, 65.1 mg, 65.2 mg, 65.3 mg, 65.4 mg, 65.5 mg, 65.6 mg, 65.7 mg, 65.8 mg, 65.9 mg, 66.0 mg, 66.1 mg, 66.2 mg, 66.3 mg, 66.4 mg, 66.5 mg, 66.6 mg, 66.7 mg, 66.8 mg, 66.9 mg, 67.0 mg, 67.1 mg, 67.2 mg, 67.3 mg, 67.4 mg, 67.5 mg, 67.6 mg, 67.7 mg, 67.8 mg, 67.9 mg, 68.0 mg, 68.1 mg, 68.2 mg, 68.3 mg, 68.4 mg, 68.5 mg, 68.6 mg, 68.7 mg, 68.8 mg, 68.9 mg, 69.0 mg, 69.1 mg, 69.2 mg, 69.3 mg, 69.4 mg, 69.5 mg, 69.6 mg, 69.7 mg, 69.8 mg, 69.9 mg, 70.0 mg, 70.1 mg, 70.2 mg, 70.3 mg, 70.4 mg, 70.5 mg, 70.6 mg, 70.7 mg, 70.8 mg, 70.9 mg, 71.0 mg, 71.1 mg, 71.2 mg, 71.3 mg, 71.4 mg, 71.5 mg, 71.6 mg, 71.7 mg, 71.8 mg, 71.9 mg, 72.0 mg, 72.1 mg, 72.2 mg, 72.3 mg, 72.4 mg, 72.5 mg, 72.6 mg, 72.7 mg, 72.8 mg, 72.9 mg, 73.0 mg, 73.1 mg, 73.2 mg, 73.3 mg, 73.4 mg, 73.5 mg, 73.6 mg, 73.7 mg, 73.8 mg, 73.9 mg, 74.0 mg, 74.1 mg, 74.2 mg, 74.3 mg, 74.4 mg, 74.5 mg, 74.6 mg, 74.7 mg, 74.8 mg, 74.9 mg, 75.0 mg, 75.1 mg, 75.2 mg, 75.3 mg, 75.4 mg, 75.5 mg, 75.6 mg, 75.7 mg, 75.8 mg, 75.9 mg, 76.0 mg, 76.1 mg, 76.2 mg, 76.3 mg, 76.4 mg, 76.5 mg, 76.6 mg, 76.7 mg, 76.8 mg, 76.9 mg, 77.0 mg, 77.1 mg, 77.2 mg, 77.3 mg, 77.4 mg, 77.5 mg, 77.6 mg, 77.7 mg, 77.8 mg, 77.9 mg, 78.0 mg, 78.1 mg, 78.2 mg, 78.3 mg, 78.4 mg, 78.5 mg, 78.6 mg, 78.7 mg, 78.8 mg, 78.9 mg, 79.0 mg, 79.1 mg, 79.2 mg, 79.3 mg, 79.4 mg, 79.5 mg, 79.6 mg, 79.7 mg, 79.8 mg, 79.9 mg, 80.0 mg, 80.1 mg, 80.2 mg, 80.3 mg, 80.4 mg, 80.5 mg, 80.6 mg, 80.7 mg, 80.8 mg, 80.9 mg, 81.0 mg, 81.1 mg, 81.2 mg, 81.3 mg, 81.4 mg, 81.5 mg, 81.6 mg, 81.7 mg, 81.8 mg, 81.9 mg, 82.0 mg, 82.0 mg, 82.1 mg, 82.2 mg, 82.3 mg, 82.4 mg, 82.5 mg, 82.6 mg, 82.7 mg, 82.8 mg, 82.9 mg, 83.0 mg, 83.1 mg, 83.2 mg, 83.3 mg, 83.4 mg, 83.5 mg, 83.6 mg, 83.7 mg, 83.8 mg, 83.9 mg, 84.0 mg, 84.1 mg, 84.2 mg, 84.3 mg, 84.4 mg, 84.5 mg, 84.6 mg, 84.7 mg, 84.8 mg, 84.9 mg, 85.0 mg, 85.0 mg, 85.1 mg, 85.2 mg, 85.3 mg, 85.4 mg, 85.5 mg, 85.6 mg, 85.7 mg, 85.8 mg, 85.9 mg, 86.0 mg, 86.1 mg, 86.2 mg, 86.3 mg, 86.4 mg, 86.5 mg, 86.6 mg, 86.7 mg, 86.8 mg, 86.9 mg, 87.0 mg, 87.1 mg, 87.2 mg, 87.3 mg, 87.4 mg, 87.5 mg, 87.6 mg, 87.7 mg, 87.8 mg, 87.9 mg, 88.0 mg, 88.1 mg, 88.2 mg, 88.3 mg, 88.4 mg, 88.5 mg, 88.6 mg, 88.7 mg, 88.8 mg, 88.9 mg, 89.0 mg, 89.0 mg, 89.1 mg, 89.2 mg, 89.3 mg, 89.4 mg, 89.5 mg, 89.6 mg, 89.7 mg, 89.8 mg, 89.9 mg, 90.0 mg, 90.1 mg, 90.2 mg, 90.3 mg, 90.4 mg, 90.5 mg, 90.6 mg, 90.7 mg, 90.8 mg, 90.9 mg, 91.0 mg, 91.1 mg, 91.2 mg, 91.3 mg, 91.4 mg, 91.5 mg, 91.6 mg, 91.7 mg, 91.8 mg, 91.9 mg, 92.0 mg, 92.0 mg, 92.1 mg, 92.2 mg, 92.3 mg, 92.4 mg, 92.5 mg, 92.6 mg, 92.7 mg, 92.8 mg, 92.9 mg, 93.0 mg, 93.1 mg, 93.2 mg, 93.3 mg, 93.4 mg, 93.5 mg, 93.6 mg, 93.7 mg, 93.8 mg, 93.9 mg, 94.0 mg, 94.1 mg, 94.2 mg, 94.3 mg, 94.4 mg, 94.5 mg, 94.6 mg, 94.7 mg, 94.8 mg, 94.9 mg, 95.0 mg, 95.1 mg, 95.2 mg, 95.3 mg, 95.4 mg, 95.5 mg, 95.6 mg, 95.7 mg, 95.8 mg, 95.9 mg, 96.0 mg, 96.1 mg, 96.2 mg, 96.3 mg, 96.4 mg, 96.5 mg, 96.6 mg, 96.7 mg, 96.8 mg, 96.9 mg, 97.0 mg, 97.1 mg, 97.2 mg, 97.3 mg, 97.4 mg, 97.5 mg, 97.6 mg, 97.7 mg, 97.8 mg, 97.9 mg, 98.0 mg, 98.1 mg, 98.2 mg, 98.3 mg, 98.4 mg, 98.5 mg, 98.6 mg, 98.7 mg, 98.8 mg, 98.9 mg, 99.0 mg, 99.1 mg, 99.2 mg, 99.3 mg, 99.4 mg, 99.5 mg, 99.6 mg, 99.7 mg, 99.8 mg, 99.9 mg, and 100.0 mg.

In certain embodiments, the therapeutically effective amount is any of about 40.0 mg, about 40.1 mg, about 40.2 mg, about 40.3 mg, about 40.4 mg, about 40.5 mg, about 40.6 mg, about 40.7 mg, about 40.8 mg, about 40.9 mg, about 41.0 mg, about 41.1 mg, about 41.2 mg, about 41.3 mg, about 41.4 mg, about 41.5 mg, about 41.6 mg, about 41.7 mg, about 41.8 mg, about 41.9 mg, about 42.0 mg, about 42.1 mg, about 42.2 mg, about 42.3 mg, about 42.4 mg, about 42.5 mg, about 42.6 mg, about 42.7 mg, about 42.8 mg, about 42.9 mg, about 43.0 mg, about 43.1 mg, about 43.2 mg, about 43.3 mg, about 43.4 mg, about 43.5 mg, about 43.6 mg, about 43.7 mg, about 43.8 mg, about 43.9 mg, about 44.0 mg, about 44.1 mg, about 44.2 mg, about 44.3 mg, about 44.4 mg, about 44.5 mg, about 44.6 mg, about 44.7 mg, about 44.8 mg, about 44.9 mg, about 45.0 mg, about 45.1 mg, about 45.2 mg, about 45.3 mg, about 45.4 mg, about 45.5 mg, about 45.6 mg, about 45.7 mg, about 45.8 mg, about 45.9 mg, about 46.0 mg, about 46.1 mg, about 46.2 mg, about 46.3 mg, about 46.4 mg, about 46.5 mg, about 46.6 mg, about 46.7 mg, about 46.8 mg, about 46.9 mg, about 47.0 mg, about 47.1 mg, about 47.2 mg, about 47.3 mg, about 47.4 mg, about 47.5 mg, about 47.6 mg, about 47.7 mg, about 47.8 mg, about 47.9 mg, about 48.0 mg, about 48.1 mg, about 48.2 mg, about 48.3 mg, about 48.4 mg, about 48.5 mg, about 48.6 mg, about 48.7 mg, about 48.8 mg, about 48.9 mg, about 49.0 mg, about 49.1 mg, about 49.2 mg, about 49.3 mg, about 49.4 mg, about 49.5 mg, about 49.6 mg, about 49.7 mg, about 49.8 mg, about 49.9 mg, about 50.0 mg, about 50.1 mg, about 50.2 mg, about 50.3 mg, about 50.4 mg, about 50.5 mg, about 50.6 mg, about 50.7 mg, about 50.8 mg, about 50.9 mg, about 51.0 mg, about 51.1 mg, about 51.2 mg, about 51.3 mg, about 51.4 mg, about 51.5 mg, about 51.6 mg, about 51.7 mg, about 51.8 mg, about 51.9 mg, about 52.0 mg, about 52.1 mg, about 52.2 mg, about 52.3 mg, about 52.4 mg, about 52.5 mg, about 52.6 mg, about 52.7 mg, about 52.8 mg, about 52.9 mg, about 53.0 mg, about 53.1 mg, about 53.2 mg, about 53.3 mg. about 53.4 mg, about 53.5 mg, about 53.6 mg, about 53.7 mg, about 53.8 mg, about 53.9 mg, about 54.0 mg, about 54.1 mg, about 54.2 mg, about 54.3 mg, about 54.4 mg, about 54.5 mg, about 54.6 mg, about 54.7 mg, about 54.8 mg, about 54.9 mg, about 55.0 mg, about 55.1 mg, about 55.2 mg, about 55.3 mg. about 55.4 mg, about 55.5 mg, about 55.6 mg, about 55.7 mg, about 55.8 mg, about 55.9 mg, about 55.0 mg, about 55.1 mg, about 55.2 mg, about 55.3 mg, about 55.4 mg, about 55.5 mg, about 55.6 mg, about 55.7 mg, about 55.8 mg, about 55.9 mg, about 56.0 mg, about 56.1 mg, about 56.2 mg, about 56.3 mg. about 56.4 mg, about 56.5 mg, about 56.6 mg, about 56.7 mg, about 56.8 mg, about 56.9 mg, about 57.0 mg, about 57.1 mg, about 57.2 mg, about 57.3 mg, about 57.4 mg, about 57.5 mg, about 57.6 mg, about 57.7 mg, about 57.8 mg, about 57.9 mg, about 58.0 mg, about 58.1 mg, about 58.2 mg, about 58.3 mg. about 58.4 mg, about 58.5 mg, about 58.6 mg, about 58.7 mg, about 58.8 mg, about 58.9 mg, about 59.0 mg, about 59.1 mg, about 59.2 mg, about 59.3 mg, about 59.4 mg, about 59.5 mg, about 59.6 mg, about 59.7 mg, about 59.8 mg, about 59.9 mg, about 60.0 mg, about 60.1 mg, about 60.2 mg, about 60.3 mg, about 60.4 mg, about 60.5 mg, about 60.6 mg, about 60.7 mg, about 60.8 mg, about 60.9 mg, about 61.0 mg, about 61.1 mg, about 61.2 mg, about 61.3 mg, about 61.4 mg, about 61.5 mg, about 61.6 mg, about 61.7 mg, about 61.8 mg, about 61.9 mg, about 62.0 mg, about 62.1 mg, about 62.2 mg, about 62.3 mg, about 62.4 mg, about 62.5 mg, about 62.6 mg, about 62.7 mg, about 62.8 mg, about 62.9 mg, about 63.0 mg, about 63.1 mg, about 63.2 mg, about 63.3 mg, about 63.4 mg, about 63.5 mg, about 63.6 mg, about 63.7 mg, about 63.8 mg, about 63.9 mg, about 64.0 mg, about 64.1 mg, about 64.2 mg, about 64.3 mg, about 64.4 mg, about 64.5 mg, about 64.6 mg, about 64.7 mg, about 64.8 mg, about 64.9 mg, about 65.0 mg, about 65.1 mg, about 65.2 mg, about 65.3 mg, about 65.4 mg, about 65.5 mg, about 65.6 mg, about 65.7 mg, about 65.8 mg, about 65.9 mg, about 66.0 mg, about 66.1 mg, about 66.2 mg, about 66.3 mg, about 66.4 mg, about 66.5 mg, about 66.6 mg, about 66.7 mg, about 66.8 mg, about 66.9 mg, about 67.0 mg, about 67.1 mg, about 67.2 mg, about 67.3 mg, about 67.4 mg, about 67.5 mg, about 67.6 mg, about 67.7 mg, about 67.8 mg, about 67.9 mg, about 68.0 mg, about 68.1 mg, about 68.2 mg, about 68.3 mg, about 68.4 mg, about 68.5 mg, about 68.6 mg, about 68.7 mg, about 68.8 mg, about 68.9 mg, about 69.0 mg, about 69.1 mg, about 69.2 mg, about 69.3 mg, about 69.4 mg, about 69.5 mg, about 69.6 mg, about 69.7 mg, about 69.8 mg, about 69.9 mg, about 70.0 mg, about 70.1 mg, about 70.2 mg, about 70.3 mg, about 70.4 mg, about 70.5 mg, about 70.6 mg, about 70.7 mg, about 70.8 mg, about 70.9 mg, about 71.0 mg, about 71.1 mg, about 71.2 mg, about 71.3 mg, about 71.4 mg, about 71.5 mg, about 71.6 mg, about 71.7 mg, about 71.8 mg, about 71.9 mg, about 72.0 mg, about 72.1 mg, about 72.2 mg, about 72.3 mg, about 72.4 mg, about 72.5 mg, about 72.6 mg, about 72.7 mg, about 72.8 mg, about 72.9 mg, about 73.0 mg, about 73.1 mg, about 73.2 mg, about 73.3 mg, about 73.4 mg, about 73.5 mg, about 73.6 mg, about 73.7 mg, about 73.8 mg, about 73.9 mg, about 74.0 mg, about 74.1 mg, about 74.2 mg, about 74.3 mg, about 74.4 mg, about 74.5 mg, about 74.6 mg, about 74.7 mg, about 74.8 mg, about 74.9 mg, about 75.0 mg, about 75.1 mg, about 75.2 mg, about 75.3 mg, about 75.4 mg, about 75.5 mg, about 75.6 mg, about 75.7 mg, about 75.8 mg, about 75.9 mg, about 76.0 mg, about 76.1 mg, about 76.2 mg, about 76.3 mg, about 76.4 mg, about 76.5 mg, about 76.6 mg, about 76.7 mg, about 76.8 mg, about 76.9 mg, about 77.0 mg, about 77.1 mg, about 77.2 mg, about 77.3 mg, about 77.4 mg, about 77.5 mg, about 77.6 mg, about 77.7 mg, about 77.8 mg, about 77.9 mg, about 78.0 mg, about 78.1 mg, about 78.2 mg, about 78.3 mg, about 78.4 mg, about 78.5 mg, about 78.6 mg, about 78.7 mg, about 78.8 mg, about 78.9 mg, about 79.0 mg, about 79.1 mg, about 79.2 mg, about 79.3 mg, about 79.4 mg, about 79.5 mg, about 79.6 mg, about 79.7 mg, about 79.8 mg, about 79.9 mg, about 80.0 mg, about 80.1 mg, about 80.2 mg, about 80.3 mg, about 80.4 mg, about 80.5 mg, about 80.6 mg, about 80.7 mg, about 80.8 mg, about 80.9 mg, about 81.0 mg, about 81.1 mg, about 81.2 mg, about 81.3 mg, about 81.4 mg, about 81.5 mg, about 81.6 mg, about 81.7 mg, about 81.8 mg, about 81.9 mg, about 82.0 mg, about 82.0 mg, about 82.1 mg, about 82.2 mg, about 82.3 mg, about 82.4 mg, about 82.5 mg, about 82.6 mg, about 82.7 mg, about 82.8 mg, about 82.9 mg, about 83.0 mg, about 83.1 mg, about 83.2 mg, about 83.3 mg, about 83.4 mg, about 83.5 mg, about 83.6 mg, about 83.7 mg, about 83.8 mg, about 83.9 mg, about 84.0 mg, about 84.1 mg, about 84.2 mg, about 84.3 mg, about 84.4 mg, about 84.5 mg, about 84.6 mg, about 84.7 mg, about 84.8 mg, about 84.9 mg, about 85.0 mg, about 85.1 mg, about 85.2 mg, about 85.3 mg, about 85.4 mg, about 85.5 mg, about 85.6 mg, about 85.7 mg, about 85.8 mg, about 85.9 mg, about 86.0 mg, about 86.1 mg, about 86.2 mg, about 86.3 mg, about 86.4 mg, about 86.5 mg, about 86.6 mg, about 86.7 mg, about 86.8 mg, about 86.9 mg, about 87.0 mg, about 87.1 mg, about 87.2 mg, about 87.3 mg, about 87.4 mg, about 87.5 mg, about 87.6 mg, about 87.7 mg, about 87.8 mg, about 87.9 mg, about 88.0 mg, about 88.1 mg, about 88.2 mg, about 88.3 mg, about 88.4 mg, about 88.5 mg, about 88.6 mg, about 88.7 mg, about 88.8 mg, about 88.9 mg, about 89.0 mg, about 89.1 mg, about 89.2 mg, about 89.3 mg, about 89.4 mg, about 89.5 mg, about 89.6 mg, about 89.7 mg, about 89.8 mg, about 89.9 mg, about 90.0 mg, about 90.1 mg, about 90.2 mg, about 90.3 mg, about 90.4 mg, about 90.5 mg, about 90.6 mg, about 90.7 mg, about 90.8 mg, about 90.9 mg, about 91.0 mg, about 91.1 mg, about 91.2 mg, about 91.3 mg, about 91.4 mg, about 91.5 mg, about 91.6 mg, about 91.7 mg, about 91.8 mg, about 91.9 mg, about 92.0 mg, about 92.1 mg, about 92.2 mg, about 92.3 mg, about 92.4 mg, about 92.5 mg, about 92.6 mg, about 92.7 mg, about 92.8 mg, about 92.9 mg, about 93.0 mg, about 93.1 mg, about 93.2 mg, about 93.3 mg, about 93.4 mg, about 93.5 mg, about 93.6 mg, about 93.7 mg, about 93.8 mg, about 93.9 mg, about 94.0 mg, about 94.1 mg, about 94.2 mg, about 94.3 mg, about 94.4 mg, about 94.5 mg, about 94.6 mg, about 94.7 mg, about 94.8 mg, about 94.9 mg, about 95.0 mg, about 95.1 mg, about 95.2 mg, about 95.3 mg, about 95.4 mg, about 95.5 mg, about 95.6 mg, about 95.7 mg, about 95.8 mg, about 95.9 mg, about 96.0 mg, about 96.1 mg, about 96.2 mg, about 96.3 mg, about 96.4 mg, about 96.5 mg, about 96.6 mg, about 96.7 mg, about 96.8 mg, about 96.9 mg, about 97.0 mg, about 97.1 mg, about 97.2 mg, about 97.3 mg, about 97.4 mg, about 97.5 mg, about 97.6 mg, about 97.7 mg, about 97.8 mg, about 97.9 mg, about 98.0 mg, about 98.1 mg, about 98.2 mg, about 98.3 mg, about 98.4 mg, about 98.5 mg, about 98.6 mg, about 98.7 mg, about 98.8 mg, about 98.9 mg, about 99.0 mg, about 99.1 mg, about 99.2 mg, about 99.3 mg, about 99.4 mg, about 99.5 mg, about 99.6 mg, about 99.7 mg, about 99.8 mg, about 99.9 mg, and about 100.0 mg.

In certain embodiments, the therapeutically effective amount is any of 40 mg to 100 mg, 40 mg to 80 mg, 40 mg to 70 mg, 40 mg to 60 mg, 40 mg to 50 mg, 50 mg to 100 mg, 50 mg to 80 mg, 50 mg to 70 mg, 50 mg to 60 mg, 60 mg to 100 mg, 60 mg to 80 mg, 60 mg to 70 mg, 70 mg to 100 mg, 70 mg to 80 mg, 80 mg to 100 mg, 80 mg to 90 mg, and 90 mg to 100 mg.

In certain embodiments, the therapeutically effective amount is any of less than 100 mg, less than 95 mg, less than 90 mg, less than 85 mg, less than 80 mg, less than 75 mg, less than 70 mg, less than 65 mg, less than 60 mg, less than 55 mg, and less than 50 mg.

In certain embodiments, the therapeutically effective amount is any of less than about 100 mg, less than about 95 mg, less than about 90 mg, less than about 85 mg, less than about 80 mg, less than about 75 mg, less than about 70 mg, less than about 65 mg, less than about 60 mg, less than about 55 mg, and less than about 50 mg.

In certain embodiments, the therapeutically effective amount is any of at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, and at least 100 mg.

In certain embodiments, the therapeutically effective amount is any of at least about 40 mg, at least about 45 mg, at least about 50 mg, at least about 55 mg, at least about 60 mg, at least about 65 mg, at least about 70 mg, at least about 75 mg, at least about 80 mg, at least about 85 mg, at least about 90 mg, at least about 95 mg, and at least about 100 mg.

In certain embodiments, the therapeutically effective amount is about 10 to 25 mg per week, which is administered to the subject in one administration every 4 weeks, one administration every 3 weeks, one administration every 2 weeks, or one administration every week. In certain embodiments, the therapeutically effective amount is about 12.5 to 20 mg per week. In certain embodiments, the therapeutically effective amount is about 12.5 mg per week. In certain embodiments, the therapeutically effective amount is about 20 mg per week.

V. Certain Dosing Regimens

In certain embodiments, described herein are methods of administering to a subject a therapeutically effective amount of the modified oligonucleotide ISIS 678354 one or more times. In certain embodiments, methods comprise administering the therapeutically effective amount at least 1, 2, 3, 4, 5, 6, 7, 8, 9 or 10 times. In certain embodiments, methods comprise administering the therapeutically effective amount once every 2 weeks. In certain embodiments, methods comprise administering the therapeutically effective amount once every 4 weeks. In certain embodiments, methods comprise administering the therapeutically effective amount once every 8 weeks. In certain embodiments, methods comprise administering the therapeutically effective amount once every 16 weeks.

In certain embodiments, methods comprise administering the therapeutically effective amount about every 1 week, about every 2 weeks, about every 3 weeks, about every 4 weeks, about every 5 weeks, about every 6 weeks, about every 7 weeks, about every 8 weeks, about every 9 weeks, about every 10 weeks, about every 11 weeks, about every 12 weeks, about every 13 weeks, about every 14 weeks, about every 15 weeks, about every 16 weeks, about every 17 weeks, about every 18 weeks, about every 19 weeks, or about every 20 weeks.

In certain embodiments, methods comprise administering the therapeutically effective amount for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months.

VI. Potency and Efficacy

In certain embodiments, described herein are methods of reducing APOCIII RNA and/or APOCIII protein in a cell or biological fluid of a human subject, wherein the methods comprise administering a therapeutically effective amount of ISIS 678354 to the subject. One may determine whether or not methods reduce APOCIII RNA and/or APOCIII protein, e.g., by detecting/quantifying a first amount of APOCIII RNA or APOCIII protein in a first biological sample obtained before administering and detecting/quantifying a second amount of APOCIII RNA or APOCIII protein in a second biological sample obtained after administering, and detecting or quantifying a reduction in APOCIII RNA or APOCIII protein by comparing the first amount to the second amount.

In certain embodiments, methods comprise reducing APOCIII RNA and/or APOCIII protein by 1-100%, or a range defined by any two of these values. In certain embodiments, methods comprise reducing APOCIII RNA and/or APOCIII protein by 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%.

In certain embodiments, methods comprise reducing APOCIII RNA or APOCIII protein by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%. In certain embodiments, the reduction in APOCIII RNA or APOCIII protein is relative to a pretreatment level.

In certain embodiments, methods comprise reducing APOCIII RNA or APOCIII protein by about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or about 95% to 100%. In certain embodiments, the reduction in APOCIII RNA or APOCIII protein is relative to a pretreatment level.

In certain embodiments, methods comprise reducing triglycerides by about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or about 95% to 100% when compared to a pretreatment level.

In certain embodiments, methods comprise reducing triglycerides to no more than about 50 mg/dL, about 100 mg/dL, about 150 mg/dL, about 200 mg/dL, about 250 mg/dL, about 300 mg/dL, about 400 mg/dL, or about 500 mg/dL. The triglyceride levels may be fasting levels.

In certain embodiments, a subject has at triglycerides of at least 500 mg/dL. In certain embodiments, the subject has triglycerides of at least 880 mg/dL. In certain embodiments, the subject has triglycerides of at least 1000 mg/dL.

Diseases and conditions described herein, for example, FCS, SHTG, and FPL, may be monitored by one or more biomarkers. The biomarkers may be one or more of, for example, liver enzymes, and/or markers associated with inflammation (e.g., vascular inflammation), vascular injury, lipid and lipoprotein metabolism, migration and infiltration of monocytes and/or macrophages. Such biomarkers include hsCRP, IL6, IL10, IL1b, TNFa, IL8, INFg, ICAM1, OxPL-apoB, MCP1, LpPLA2 activity, and fibrinogen. In certain embodiments, amelioration of FCS, SHTG, or FPL is determined by modulation of one or more biomarkers following administration of ISIS 678354.

Diseases and conditions described herein, for example, FCS, SHTG, and FPL, may be monitored by one or more symptoms. The symptom may be chylomicronemia, abdominal pain, repetitive colicky pain, physical fatigue, difficulty thinking, diarrhea, recurrent acute pancreatitis, eruptive xanthomas, lipemia retinalis, or hepatosplenomegaly, or a combination thereof. In certain embodiments, amelioration of FCS, SHTG, or FPL is indicated by modulation of one or more symptoms.

In certain embodiments, methods comprise administering ISIS 678354 to a subject and detecting or quantifying an amount of APOCIII RNA or APOCIII protein in a cell or a biological fluid of the subject. In certain embodiments, methods comprise detecting/quantifying a first amount of APOCIII RNA or APOCIII protein in a first biological sample obtained before administering and detecting/quantifying a second amount of APOCIII RNA or APOCIII protein in a second biological sample obtained after administering, and detecting or quantifying a reduction in APOCIII RNA or APOCIII protein by comparing the first amount to the second amount. In certain embodiments, the second biological sample is obtained less than about 24 hours after administering. In certain embodiments, the second biological sample is obtained less than about 1 week after administering. In certain embodiments, the second biological sample is obtained about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, or about 18 weeks after administering. In certain embodiments, methods comprise increasing or decreasing the dose after comparing the first amount to the second amount. In certain embodiments, methods comprise administering more frequently or less frequently after comparing the first amount to the second amount.

### Overview of Familial Chylomicronemia Syndrome

Familial Chylomicronemia Syndrome (FCS) is an inherited disease characterized by severe hypertriglyceridemia and chylomicronemia. It is a rare autosomal recessive disease that can be diagnosed either in childhood or adulthood.

FCS is characterized by frequent and severe abdominal pain, repetitive colicky pain, repeated episodes of potentially fatal acute pancreatitis, and in children, can result in a failure to thrive (Brunzell J D. Familial Lipoprotein Lipase Deficiency. In GeneReviews edited by Adam M P Pagon R A, Bird T D, et al. 1999-2011. Seattle, WA: University of Washington, Seattle; Tremblay K, Methot J, Brisson D, et al. J Clin Lipidol 2011; 5: 37-44). Physical examination frequently reveals eruptive xanthomas, lipemia retinalis and hepatosplenomegaly, and plasma from patients appears latescent, interfering with determination of other laboratory parameters. Fasting plasma TG levels in FCS patients are typically 10-fold to 100-fold above normal (1,500 to 15,000 mg/dL), despite extreme dietary fat restriction (20 g or approximately 15-20% of daily calorie intake.

Patients with FCS often present in infancy or childhood with recurrent episodes of abdominal pain or pancreatitis, eruptive xanthomas or hepatomegaly. The diagnosis of FCS is then established by genotyping or confirmation of very low or absent lipoprotein lipase (LPL) enzyme activity in post-heparin plasma.

Patients with FCS carry a heavy burden of medical complications, the most serious being an extreme risk of recurrent and potentially fatal pancreatitis. Due to the recurrent episodes of acute pancreatitis, these patients may also develop chronic pancreatitis and signs of exocrine or endocrine pancreatic insufficiency, including diabetes mellitus (Gaudet D, Methot J, Dery S, et al. Gene Ther 2013; 20: 361-369). While the pathophysiology underlying chylomicron-related pancreatitis has not been completely elucidated, one hypothesis is that large chylomicrons lodged in pancreatic capillaries are exposed to pancreatic lipase, resulting in release of free fatty acids through the hydrolysis of chylomicron-associated TGs. High concentrations of free fatty acids are thought to damage pancreatic cells leading to emergent pancreatitis (Yang F, Wang Y, Sternfeld L, et al. Acta Physiol (Oxf) 2009; 195: 13-28; Berglund L, Brunzell J D, Goldberg A C, et al. J Clin Endocrinol Metab 2012; 97: 2969-2989).

FCS significantly affects patients' HRQoL. Bloating, generalized abdominal pain, asthenia, anxiety about potential painful attacks and overall health, difficulty concentrating and “brain fog” are commonly reported symptoms of FCS. The psychosocial burden of FCS is also increased by dietary fat restriction and overall interference with social interactions and ability to work (Davidson M, Stevenson M, Hsieh A, et al. Expert Rev Cardiovasc Ther 2017; 15: 415-423; Gelrud A, Williams K R, Hsieh A, et al. Expert Rev Cardiovasc Ther 2017; 15: 879-887; Davidson M, Stevenson M, Hsieh A, et al. J Clin Lipidol 2018; 12: 898-907.e892; Fox R S, Peipert J D, Llonch M V, et al. Expert Rev Cardiovasc Ther 2020: 1-8).

The etiology of extreme hypertriglyceridemia in FCS is considered to be ineffective TG clearance, due to an extremely low level of LPL activity. LPL normally functions to hydrolyze TGs in chylomicrons along the luminal surface of capillaries, mainly in heart, skeletal muscle, and adipose tissue, promoting TG clearance from the circulation. LPL is regulated by a number of key genes, and loss-of-function mutations in one of these genes, or the LPL gene itself, results in FCS (Surendran R P, Visser M E, Heemelaar S, et al. J Intern Med 2012; 272: 185-196). In addition to loss of function mutations, null mutations, and nonsense mutations in the LPL gene, other genes currently identified in FCS patients, and known to directly influence LPL activity include: apolipoprotein C-II (APOC2) a cofactor for LPL (Schuster K B, Wilfert W, Evans D, et al. Clin Chim Acta 2011; 412: 240-244); apolipoprotein A-V (APOA5) (Schaap F G, Rensen P C, Voshol P J, et al. J Biol Chem 2004; 279: 27941-27947); lipase maturation Factor 1 (LMF1), a transmembrane protein involved in LPL maturation (Doolittle M H, Neher S B, Ben-Zeev O, et al J Biol Chem 2009; 284: 33623-33633), glycosylphosphotidylinositol-anchored HDL-binding protein 1 (GP1HBP1), a capillary endothelial cell protein that provides a platform for LPL-mediated processing of chylomicrons (Beigneux A P, Davies B S, Gin P, et al. Cell Metab 2007; 5: 279-291).

### Overview of Familial Partial Lipodystrophy

Familial Partial Lipodystrophy refers to a familial disorder characterized by selective, progressive loss of body fat (adipose tissue) from various areas of the body. Individuals with FPL often have reduced subcutaneous fat in the arms and legs, and the head and trunk regions may or may not have loss of fat. Conversely, affected individuals may also have excess subcutaneous fat accumulation in other areas of the body, especially the neck, face and intra-abdominal regions. In many cases, adipose tissue loss begins during puberty. FPL can be associated with a variety of metabolic abnormalities. FPL is associated with certain metabolic complications. These complications can include an inability to metabolize glucose, elevated levels of triglycerides, and diabetes. Six different subtypes of FPL have been identified. Each subtype is caused by a mutation in a different gene. Four forms of FPL are inherited as autosomal dominant traits; one form is inherited as an autosomal recessive trait. The mode of inheritance of FPL, Kobberling variety is unknown.

Types of FPL include FPL2 (Dunnigan variety), FPL1 (Kobberling variety), FPL3 (PPARG Mutations), FPL4 (PLIN1 Mutations), FPL5 (AKT2 Mutations), and Autosomal Recessive FPL (Type 6, CIDEC mutation).

### Overview of Severe Hypertriglyceridemia

As used herein, Severe Hypertriglyceridemia (SHTG) refers to a condition in which a subject has triglycerides at a level at which chylomicrons appear in the blood. In certain embodiments, a subject has at triglycerides of at least 500 mg/dL. SHTG may be acquired or familial. For example, a subject having FCS or FPL may also be diagnosed as having SHTG. In certain embodiments, the subject has triglycerides of at least 880 mg/dL. In certain embodiments, the subject has triglycerides of at least 1000 mg/dL. SHTG may arise in subjects having obesity, a history of alcohol abuse, and/or diabetes. SHTG may arise due to a combination of weak genetic factors combined with secondary factors such as certain medications (e.g., oral estrogens, glycocorti-costeroids, protease inhibitors, some antihypertensive medications such as hydrochlorothiazide, and nonselective beta-blockers, retinoic acid (isotretinoin), tamoxifen, raloxifen, cyclosporin, sirolimus, bile acid-binding resins, and antipsychotic medications including clozapine and olanzapine) or metabolic disorders (e.g., obesity, diabetes, hypothyroidism, or kidney disease), or from genetic factors alone. Patients having SHTG are at risk for acute pancreatitis. See, e.g., Cybulska, B. et al., Kardiologia Polska 2013; 71, 10:1007-1012.

### Assessing Efficacy of ISIS 678354

In certain embodiments, methods described herein are sufficiently effective to ameliorate at least one symptom of FCS in a human subject. In certain embodiments, the at least one symptom is severe elevations in chylomicrons. In certain embodiments, the at least one symptom is extremely elevated TG levels (always reaching well above 1000 mg/dL and not infrequently rising as high as 10,000 mg/dL or more). In certain embodiments, the at least one symptom is episodes of abdominal pain. In certain embodiments, the at least one symptom is recurrent acute pancreatitis. In certain embodiments, the at least one symptom is repetitive colicky pain. In certain embodiments, the at least one symptom is eruptive xanthomas. In certain embodiments, the at least one symptom is hepatosplenomegaly. In certain embodiments, the at least one symptom is physical fatigue. In certain embodiments, the at least one symptom is difficulty thinking. In certain embodiments, the at least one symptom is diarrhea. In certain embodiments, the at least one symptom is difficulty thinking. In certain embodiments, the at least one symptom is recurrent acute pancreatitis. In certain embodiments, the at least one symptom is lipemia retinalis. In certain embodiments, the at least one symptom is a combination of any one of severe chylomicronemia, severe hypertriglyceridemia, frequent and severe abdominal pain, repetitive colicky pain, physical fatigue, difficulty thinking, diarrhea, recurrent acute pancreatitis, eruptive xanthomas, lipemia retinalis, and hepatosplenomegaly.

In certain embodiments, methods described herein are sufficiently effective to ameliorate any one of severe chylomicronemia, severe hypertriglyceridemia, frequent and severe abdominal pain, repetitive colicky pain, physical fatigue, difficulty thinking, diarrhea, recurrent acute pancreatitis, eruptive xanthomas, lipemia retinalis, and hepatosplenomegaly.

In certain embodiments, methods described herein are sufficiently effective to ameliorate at least one symptom of FCS in a human subject as assessed by a clinically relevant test, score or scale. In certain embodiments, the clinical relevant scale is the Patient Global Impression of Severity (PGIS) Scale. In certain embodiments, the clinical relevant scale is the Patient Global Impression of Change (PGIC) Scale. In certain embodiments, the clinically relevant test is fasting triglyceride levels. In certain embodiments, the clinically relevant test is fasting apoB-48 levels. In certain embodiments, the clinically relevant test, score or scale is a decrease the adjudicated pancreatitis event rate in patients with ≥2 events in 5 years prior to enrollment. In certain embodiments, the clinically relevant test, score or scale is number of emergency room (ER) visits, incidence of all-cause hospitalizations, and total inpatient days. In certain embodiments, health-related quality of life is measured by the PROMIS 29+2 Profile vs. 2.1 questionnaire.

In certain embodiments, methods described herein are sufficiently tolerable in safety and tolerability assessments, including adverse events, clinical laboratory tests, ECGs, use of concomitant medications, and independently adjudicated events rates of Major Cardiovascular Events (MACE) for ISIS 678354 as compared to placebo.

VII. Certain Combination Therapies

In certain embodiments, methods comprise co-administering ISIS 678354 with at least one other pharmaceutical agent. In certain embodiments, the at least one other pharmaceutical agent ameliorates HD or a symptom thereof. In certain embodiments, ISIS 678354 is co-administered with the at least one other pharmaceutical agent to produce a combinational effect. In certain embodiments, ISIS 678354 is co-administered with the at least one other pharmaceutical agent to produce a synergistic effect.

In certain embodiments, ISIS 678354 and the at least one other pharmaceutical agent are administered at the same time. In certain embodiments, ISIS 678354 and the at least one other pharmaceutical agent are administered at different times. In certain embodiments, ISIS 678354 and the at least one other pharmaceutical agent are prepared together in a single formulation. In certain embodiments, ISIS 678354 and the at least one other pharmaceutical agent are administered are prepared separately.

### EXAMPLES

The following examples illustrate certain embodiments of the present disclosure and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif And, for example, where a particular high-affinity modification appears at a particular position, other high-affinity modifications at the same position are considered suitable, unless otherwise indicated.

### Example 1: Clinical Protocol Including Dosing Parameters, Efficacy Endpoints and Safety Endpoints

A randomized, double blind, placebo controlled, Phase 3 study is carried out with Compound No. 678354 administered subcutaneously to patients with Familial Chylomicronemia Syndrome (FCS).

Cohort A (consisting of approximately n=30) is randomized 2:1 to receive either 50 mg of Compound No. 678354 or a matching volume of placebo (0.5 mL) subcutaneously every 4 weeks for Weeks 1-25. Starting at week 29, Cohort A receives an escalated dose of 80 mg of Compound No. 678354, or continues with a matching volume of placebo (0.8 mL) once every 4 weeks for Weeks 29-49, for a total of 13 doses. Cohort B (consisting of approximately n=30) is randomized 2:1 to receive either 80 mg of Compound No. 678354, or a matching volume of placebo (0.8 mL) subcutaneously every 4 weeks for Weeks 1-49 for a total of 13 doses.

Routine blood chemistry and urine samples are taken after fasting for 10-12 hours. Efficacy parameters tested include percent change in fasting triglycerides (TG) from baseline (defined as the average of the Day 1 pre-dose assessment and the last measurement prior to Day 1) at 6 months (the average of Weeks 23, 25 and 27) compared to placebo, and at 12 months (average of Weeks 51 and 53) compared to placebo. Patient inclusion criteria includes a fasting triglyceride level of ≥880 mg/dL (10 mmol/L) before treatment. Post-treatment evaluation includes determining the proportion of patients who achieve ≥40% reduction in fasting TG from baseline, the proportion of patients who achieve fasting TG ≤750 mg/dL (8.4 mmol/L), the proportion of patients who achieve ≥70% reduction in fasting TG from baseline, and the proportion of patients who achieve fasting TG ≤500 mg/dL (5.7 mmol/L). In addition, evaluation criteria include percent change in fasting ApoB-48 from baseline, as well as determining the adjudicated acute pancreatitis event rate. Additionally, pharmacokinetic analysis includes determination of trough (pre-dose) and post-treatment plasma concentrations of Compound No. 678354 in all patients who receive the modified oligonucleotide.

Safety parameters tested include platelet counts, renal function testing, liver function testing. All patients have liver function monitored every 14 days (±2 days) for the first 3 months of the treatment period, and monthly testing thereafter during the treatment period. Liver function tests include monitoring for appearance of signs of hepatic injury (jaundice, fatigue, nausea, vomiting, right upper quadrant pain or tenderness, fever, rash, abnormal bleeding or bruising, or eosinophilia >ULN), followed by liver enzyme and bilirubin evaluation in the event of signs of hepatic injury. All patients have renal function tests monitored every 14 days (±2 days) for the first 3 months of the treatment period, and monthly testing thereafter during the treatment period. Renal function surveillance includes serum creatinine and urinalysis including UACR (Urine Albumin/Creatinine Ratio), UPCR (Urine Protein/Creatinine Ratio), urinary red blood cells (RBC). All patients have platelet counts monitored at least every 14 days (±2 days) for the duration of the study treatment period, and at all post-treatment follow-up visits. All patients are evaluated for occurrence of bleeding events continuously after the start of treatment (Day 1) up to the end of the follow-up period.

After the 53-week treatment and assessment period, there is a 13-week post-treatment evaluation period, where the patients may elect to enroll in an OLE (Open Label Extension) study pending study approval of this proposed study by the IRB/IEC and the appropriate regulatory authority. The treatment is therapeutically effective in ameliorating or treating FCS (as observed by amelioration of one or more symptoms, or by modulation of one or more biomarkers).

### Example 2: Clinical Protocol Including Dosing Parameters, Efficacy Endpoints and Safety Endpoints

A randomized, double blind, placebo controlled study is carried out with Compound No. 678354 administered subcutaneously to patients with Familial Partial Lypodystrophy (FPL).

Cohort A (consisting of approximately n=30) is randomized 2:1 to receive either 50 mg of Compound No. 678354 or a matching volume of placebo (0.5 mL) subcutaneously every 4 weeks for Weeks 1-25. Starting at week 29, Cohort A receives an escalated dose of 80 mg of Compound No. 678354, or continues with a matching volume of placebo (0.8 mL) once every 4 weeks for Weeks 29-49, for a total of 13 doses. Cohort B (consisting of approximately n=30) is randomized 2:1 to receive either 80 mg of Compound No. 678354, or a matching volume of placebo (0.8 mL) subcutaneously every 4 weeks for Weeks 1-49 for a total of 13 doses.

Routine blood chemistry and urine samples are taken after fasting for 10-12 hours. Efficacy parameters tested include percent change in fasting triglycerides (TG) from baseline (defined as the average of the Day 1 pre-dose assessment and the last measurement prior to Day 1) at 6 months (the average of Weeks 23, 25 and 27) compared to placebo, and at 12 months (average of Weeks 51 and 53) compared to placebo. Patient inclusion criteria includes a fasting triglyceride level of ≥880 mg/dL (10 mmol/L) before treatment. Post-treatment evaluation includes determining the proportion of patients who achieve ≥40% reduction in fasting TG from baseline, the proportion of patients who achieve fasting TG ≤750 mg/dL (8.4 mmol/L), the proportion of patients who achieve ≥70% reduction in fasting TG from baseline, and the proportion of patients who achieve fasting TG ≤500 mg/dL (5.7 mmol/L). In addition, evaluation criteria include percent change in fasting ApoB-48 from baseline, as well as determining the adjudicated acute pancreatitis event rate. Additionally, pharmacokinetic analysis includes determination of trough (pre-dose) and post-treatment plasma concentrations of Compound No. 678354 in all patients who receive the modified oligonucleotide.

Safety parameters tested include platelet counts, renal function testing, liver function testing. All patients have liver function monitored every 14 days (±2 days) for the first 3 months of the treatment period, and monthly testing thereafter during the treatment period. Liver function tests include monitoring for appearance of signs of hepatic injury (jaundice, fatigue, nausea, vomiting, right upper quadrant pain or tenderness, fever, rash, abnormal bleeding or bruising, or eosinophilia >ULN), followed by liver enzyme and bilirubin evaluation in the event of signs of hepatic injury. All patients have renal function tests monitored every 14 days (±2 days) for the first 3 months of the treatment period, and monthly testing thereafter during the treatment period. Renal function surveillance includes serum creatinine and urinalysis including UACR (Urine Albumin/Creatinine Ratio), UPCR (Urine Protein/Creatinine Ratio), urinary red blood cells (RBC). All patients have platelet counts monitored at least every 14 days (±2 days) for the duration of the study treatment period, and at all post-treatment follow-up visits. All patients are evaluated for occurrence of bleeding events continuously after the start of treatment (Day 1) up to the end of the follow-up period.

After the 53-week treatment and assessment period, there is a 13-week post-treatment evaluation period.

The treatment is therapeutically effective in ameliorating or treating FPL (as observed by amelioration of one or more symptoms, or by modulation of one or more biomarkers).

### Example 3: Clinical Protocol Including Dosing Parameters, Efficacy Endpoints and Safety Endpoints

A randomized, double blind, placebo controlled study is carried out with Compound No. 678354 administered subcutaneously to patients with Severe Hypertriglyceridemia (SHTG).

Cohort A (consisting of approximately n=30) is randomized 2:1 to receive either 50 mg of Compound No. 678354 or a matching volume of placebo (0.5 mL) subcutaneously every 4 weeks for Weeks 1-25. Starting at week 29, Cohort A receives an escalated dose of 80 mg of Compound No. 678354, or continues with a matching volume of placebo (0.8 mL) once every 4 weeks for Weeks 29-49, for a total of 13 doses. Cohort B (consisting of approximately n=30) is randomized 2:1 to receive either 80 mg of Compound No. 678354, or a matching volume of placebo (0.8 mL) subcutaneously every 4 weeks for Weeks 1-49 for a total of 13 doses.

Routine blood chemistry and urine samples are taken after fasting for 10-12 hours. Efficacy parameters tested include percent change in fasting triglycerides (TG) from baseline (defined as the average of the Day 1 pre-dose assessment and the last measurement prior to Day 1) at 6 months (the average of Weeks 23, 25 and 27) compared to placebo, and at 12 months (average of Weeks 51 and 53) compared to placebo. Patient inclusion criteria includes a fasting triglyceride level of ≥500 mg/dL before treatment. Post-treatment evaluation includes determining the proportion of patients who achieve ≥40% reduction in fasting TG from baseline, the proportion of patients who achieve fasting TG ≤500 mg/dL, the proportion of patients who achieve ≥70% reduction in fasting TG from baseline, and the proportion of patients who achieve fasting TG ≤135 mg/dL. In addition, evaluation criteria include percent change in fasting ApoB-48 from baseline, as well as determining the adjudicated acute pancreatitis event rate. Additionally, pharmacokinetic analysis includes determination of trough (pre-dose) and post-treatment plasma concentrations of Compound No. 678354 in all patients who receive the modified oligonucleotide.

Safety parameters tested include platelet counts, renal function testing, liver function testing. All patients have liver function monitored every 14 days (±2 days) for the first 3 months of the treatment period, and monthly testing thereafter during the treatment period. Liver function tests include monitoring for appearance of signs of hepatic injury (jaundice, fatigue, nausea, vomiting, right upper quadrant pain or tenderness, fever, rash, abnormal bleeding or bruising, or eosinophilia >ULN), followed by liver enzyme and bilirubin evaluation in the event of signs of hepatic injury. All patients have renal function tests monitored every 14 days (±2 days) for the first 3 months of the treatment period, and monthly testing thereafter during the treatment period. Renal function surveillance includes serum creatinine and urinalysis including UACR (Urine Albumin/Creatinine Ratio), UPCR (Urine Protein/Creatinine Ratio), urinary red blood cells (RBC). All patients have platelet counts monitored at least every 14 days (±2 days) for the duration of the study treatment period, and at all post-treatment follow-up visits. All patients are evaluated for occurrence of bleeding events continuously after the start of treatment (Day 1) up to the end of the follow-up period.

After the 53-week treatment and assessment period, there is a 13-week post-treatment evaluation period.

The treatment is therapeutically effective in ameliorating or treating SHTG (as observed by amelioration of one or more symptoms, or by modulation of one or more biomarkers).

1-35. (canceled)

36. A method of ameliorating FCS, reducing APOCIII RNA, or reducing APOCIII protein, in a human subject in need thereof, the method comprising subcutaneously administering about once every 4 weeks to the human subject 50 mg or about 50 mg of a compound according to the following chemical structure: [symbol]
(SEQ ID NO: 3), or a salt thereof, wherein at least one symptom of FCS is ameliorated.

37. A method of ameliorating FCS, reducing APOCIII RNA, or reducing APOCIII protein, in a human subject in need thereof, the method comprising subcutaneously administering about once every 4 weeks to the human subject 80 mg or about 80 mg of a compound according to the following chemical structure: [symbol]
(SEQ ID NO: 3), or a salt thereof, wherein at least one symptom of FCS is ameliorated.

38. The method of claim 36 or claim 37 wherein the compound is a sodium salt or a potassium salt.

39. A method of ameliorating FCS, reducing APOCIII RNA, or reducing APOCIII protein, in a human subject in need thereof, the method comprising intrathecally administering about once every 4 weeks to the human subject 50 mg or about 50 mg of a compound according to the following chemical structure: [symbol]
(SEQ ID NO: 3), wherein at least one symptom of FCS is ameliorated.

40. A method of ameliorating FCS, reducing APOCIII RNA, or reducing APOCIII protein, in a human subject in need thereof, the method comprising subcutaneously administering about once every 4 weeks to the human subject 80 mg or about 80 mg of a compound according to the following chemical structure: [symbol]
(SEQ ID NO: 3), wherein at least one symptom of FCS is ameliorated.

41. A method of ameliorating FCS, reducing APOCIII RNA, or reducing APOCIII protein in a human subject in need thereof, the method comprising subcutaneously administering about once every 4 weeks to the human subject 50 mg or about 50 mg of a compound, wherein the compound comprises a modified oligonucleotide having the following chemical notation (5′ to 3′): AₑₛGₑₛ^{m}CₑₛTₑₛTₑₛ^{m}C_{ds}T_{ds}T_{ds}G_{ds}T_{ds}^{m}C_{ds}^{m}C_{ds}A_{ds}G_{ds}^{m}C_{ds}TₑₛTₑₛTₑₛAₑₛTₑ (SEQ ID NO: 3), wherein,
A=an adenine nucleobase,
mC=a 5-methyl cytosine nucleobase,
G=a guanine nucleobase,
T=a thymine nucleobase,
e=a 2′-OCH₂CH₂OCH₃ modified ribosyl sugar moiety,
d=a 2′-β-D-deoxyribosyl sugar moiety, and
s=a phosphorothioate internucleoside linkage,
wherein the modified oligonucleotide has a 5′-trishexylamino-(THA)-C₆GalNAc₃ endcap, represented by the structure below, wherein the phosphate group is attached to the 5′-oxygen atom of the 5′-nucleoside: 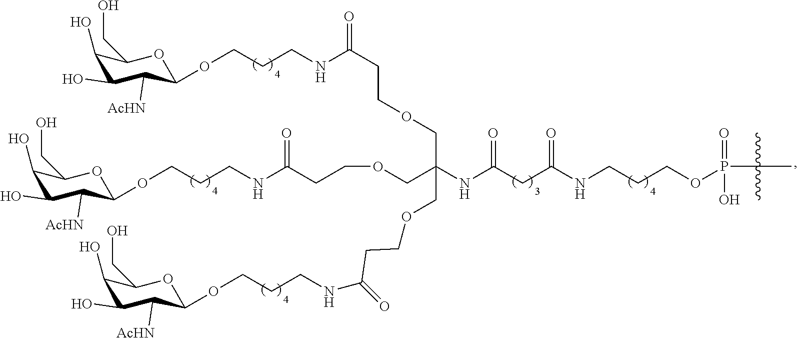
and wherein at least one symptom of FCS is ameliorated.

42. A method of ameliorating FCS, reducing APOCIII RNA, or reducing APOCIII protein in a human subject in need thereof, the method comprising subcutaneously administering about once every 4 weeks to the human subject 80 mg or about 80 mg of a compound, wherein the compound comprises a modified oligonucleotide having the following chemical notation (5′ to 3′): AₑₛGₑₛ^{m}CₑₛTₑₛTₑₛ^{m}C_{ds}T_{ds}T_{ds}G_{ds}T_{ds}^{m}C_{ds}^{m}C_{ds}A_{ds}G_{ds}^{m}C_{ds}TₑₛTₑₛTₑₛAₑₛTₑ (SEQ ID NO: 3); wherein,
A=an adenine nucleobase,
mC=a 5-methyl cytosine nucleobase,
G=a guanine nucleobase,
T=a thymine nucleobase,
e=a 2′-OCH₂CH₂OCH₃ modified ribosyl sugar moiety,
d=a 2′-β-D-deoxyribosyl sugar moiety, and
s=a phosphorothioate internucleoside linkage,
wherein the modified oligonucleotide has a 5′-trishexylamino-(THA)-C₆GalNAc₃ endcap, represented by the structure below, wherein the phosphate group is attached to the 5′-oxygen atom of the 5′-nucleoside: 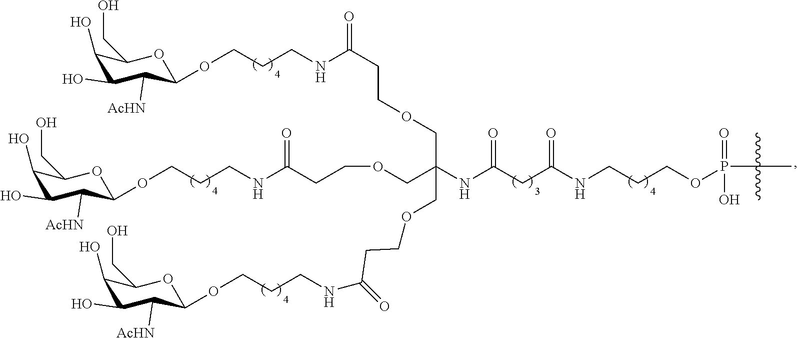
and wherein at least one symptom of FCS is ameliorated.

43-46. (canceled)

47. The method of any one of claims 36-37, and 39-42, wherein the at least one symptom comprises severe elevations in chylomicrons and extremely elevated TG levels (always reaching well above 1000 mg/dL and not infrequently rising as high as 10,000 mg/dL or more) with episodes of abdominal pain, physical fatigue, difficulty thinking, diarrhea, recurrent acute pancreatitis, eruptive cutaneous xanthomata, and hepatosplenomegaly, or a combination thereof.

48-95. (canceled)
